(19) United States
(12) Patent Application Publication
(10) Pub. No.: US 2024/0269212 A1
(43) Pub. Date: Aug. 15, 2024

(54) HEMP EXTRACT FOR TREATMENT OF PAIN, CANCER AND EPILEPSY IN ANIMALS

(71) Applicant: Portland Technology Holdings LLC., South Portland, ME (US)

(72) Inventors: Joseph WAKSHLAG, South Portland, ME (US); Amanda HOWLAND, South Portland, ME (US); Christian KJAER, South Portland, ME (US)

(21) Appl. No.: 18/625,711

(22) Filed: Apr. 3, 2024

Related U.S. Application Data
(63) Continuation of application No. PCT/US2022/046583, filed on Oct. 13, 2022.
(60) Provisional application No. 63/269,308, filed on Mar. 14, 2022.
(60) Provisional application No. 63/262,457, filed on Oct. 13, 2021.

(51) Int. Cl. A61K 36/185; A23K 10/30; A23K 50/48; A61K 31/00; A61K 47/44

(52) U.S. Cl. CPC A61K 36/3482; A23K 10/30; A23K 50/48; A61K 31/658; A61K 47/44

(57) ABSTRACT

The present disclosure relates to pharmaceutical compositions comprising hemp extract and a carrier. The present disclosure also relates to dosage forms and methods of treatment using the pharmaceutical compositions.

### CROSS-REFERENCE TO RELATED APPLICATIONS

This application is a continuation of International Application No. PCT/US2022/046583, filed on Oct. 13, 2022, which claims the benefit of U.S. Provisional Application No. 63/262,457, filed Oct. 13, 2021 and U.S. Provisional Application No. 63/269,308, filed Mar. 14, 2022, the entire disclosure of each of which is hereby incorporated by reference.

### BACKGROUND

A recent survey by the American Holistic Veterinary Medical Association revealed that almost 60% of people who buy hemp products online use these products for their dogs. Industrial hemp products that are low in THC (0.3%) and higher in other cannabinoids are reported to have health benefits including analgesic, anti-inflammatory, anti-anxiolytic, and anti-epileptic; and are legal according to the industrial hemp act. There are numerous on-line companies selling hemp products including CBD, CBG, and other oils claiming they are safe and effective for various medical conditions in both pets and people. There is very little published data to support these claims and no data to indicate the safety of using CBD, CBG, and other oils concurrently with chemotherapy in veterinary patients. In the absence of an optimal treatment for these dogs for pain, epilepsy, or cancer, other potentially efficacious pharmacological agents, including cannabinoids, are often sought.

### SUMMARY

The disclosure provides a pharmaceutical composition comprising hemp extract and a carrier, wherein the hemp extract comprises: cannabigerol; and cannbigerolic acid; wherein the ratio of cannabigerol to cannabigerolic acid is about 0.2:1 to about 1:0.2.

The disclosure also provides a pharmaceutical composition comprising hemp extract and a carrier, wherein the hemp extract comprises:
cannabigerolic acid;
cannabigerol;
cannabidiol;
cannabidiolic acid;
Δ9-tetrahydrocannabinol; and
cannabichromene;

wherein the ratio of cannabigerol to cannabigerolic acid is about 0.6:1 to about 1:0.6.

In some embodiments, the pharmaceutical composition further comprises:
α-pinene;
β-myrcene;
β-pinene;
δ-limonene;
linalool;
β-caryophyllene;
α-humulene;
nerolidol;
guaiol;
caryophyllene oxide; and
α-bisabolol.

In some embodiments, the concentration of Δ9-tetrahydrocannabinol is insufficient to produce a psychotropic effect. In some embodiments, the ratio of Δ9-tetrahydrocannabinol to the other cannabinoids is about 1:25. In some embodiments, the concentration of Δ9-tetrahydrocannabinol is less than about 10 mg/mL. In some embodiments, the concentration of Δ9-tetrahydrocannabinol is less than about 5 mg/mL. In some embodiments, the concentration of Δ9-tetrahydrocannabinol is less than about 3 mg/mL. In some embodiments, the concentration of Δ9-tetrahydrocannabinol is less than about 2 mg/mL. In some embodiments, the concentration of Δ9-tetrahydrocannabinol is less than about 1 mg/mL. In some embodiments, the concentration of Δ9-tetrahydrocannabinol is less than about 0.5 mg/mL. In some embodiments, the concentration of Δ9-tetrahydrocannabinol is less than about 0.3 mg/mL. In some embodiments, the concentration of Δ9-tetrahydrocannabinol is less than about 0.2 mg/mL. In some embodiments, the concentration of Δ9-tetrahydrocannabinol is less than about 0.1 mg/mL. In some embodiments, the concentration of Δ9-tetrahydrocannabinol is less than about 0.01 mg/mL. In some embodiments, the concentration of Δ9-tetrahydrocannabinol is about 0 mg/mL. In some embodiments, the concentration of Δ9-tetrahydrocannabinol is undetectable.

In some embodiments, the concentration of Δ9-tetrahydrocannabinol is less than about 1%. In some embodiments, the concentration of Δ9-tetrahydrocannabinol is less than about 0.5%. In some embodiments, the concentration of Δ9-tetrahydrocannabinol is less than about 0.3%. In some embodiments, the concentration of Δ9-tetrahydrocannabinol is less than about 0.2%. In some embodiments, the concentration of Δ9-tetrahydrocannabinol is less than about 0.1%. In some embodiments, the concentration of Δ9-tetrahydrocannabinol is less than about 0.01%.

In some embodiments, the hemp extract comprises:
about 1-10 mg/mL of cannabigerol;
about 1-10 mg/mL of cannabigerolic acid;
less than 0.1 mg/mL Δ9-tetrahydrocannabinol; and
about 0.1-0.4 mg/mL cannabichromene.

In some embodiments, the hemp extract comprises:
about 5 mg/mL of cannabigerol;
about 5 mg/mL of cannabigerolic acid;
less than 0.1 mg/mL Δ9-tetrahydrocannabinol; and
about 0.27 mg/mL cannabichromene.

In some embodiments, the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

In some embodiments, the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

In some embodiments, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In some embodiments, the hemp extract comprises:
about 5% of cannabigerol;
about 5% of cannabigerolic acid;
less than 0.1% Δ9-tetrahydrocannabinol; and about 0.27% cannabichromene.

In some embodiments, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

In some embodiments, the hemp extract comprises:
camphene;
β-ocimene;
eucalyptol;
isopulegol; and/or
nerolidol.

In some embodiments, the hemp extract comprises:
about 0.02% camphene;
about 0.02-0.03% β-ocimene;
about 0.02-0.05% eucalyptol;
about 0.02% isopulegol; and/or about 0.02-0.04% nerolidol.

In some embodiments, the composition is formulated in a carrier. In some embodiments,
the carrier is selected from the group consisting of linseed oil, olive oil, fish oil, salmon oil, coconut oil, catnip oil, sesame oil, MCT oil, and grapeseed oil. In some embodiments, the carrier comprises grapeseed oil. In some embodiments, the carrier comprises catnip oil. In some embodiments, the carrier comprises sesame oil.

In some embodiments, the composition comprises lecithin. In some embodiments, the lecithin is sunflower lecithin. In some embodiments, the sunflower lecithin is up to 40%.

In some embodiments, the composition further comprises NF-971P. In some embodiments, the NF-971P is up to 2% weight/volume ratio.

In some embodiments, the hemp extract comprises:
cannabigerolic acid;
cannabigerol;
cannabidiol;
cannabidiolic acid;
Δ9-tetrahydrocannabinol; and
cannabichromene.

In some embodiments, the hemp extract comprises:
about 1-10 mg/mL of cannabigerolic acid;
about 1-10 mg/mL of cannabigerol;
about 0.1-0.5 mg/mL cannabidiol;
about 0.1-0.5 mg/mL cannabidiolic acid;
less than 0.1 mg/mL Δ9-tetrahydrocannabinol; and
about 0.1-0.4 mg/mL cannabichromene.

In some embodiments, the hemp extract comprises:
about 10-100 mg/mL of cannabigerol;
about 10-100 mg/mL of cannabigerolic acid;
about 1-5 mg/mL cannabidiol;
about 1-5 mg/mL cannabidiolic acid;
less than 1 mg/mL Δ9-tetrahydrocannabinol; and
about 1-4 mg/mL cannabichromene.

In some embodiments, the hemp extract comprises:
about 1-10% of cannabigerol;
about 1-10% of cannabigerolic acid;
about 0.1-0.5% cannabidiol;
about 0.1-0.5% cannabidiolic acid;
less than 0.1% Δ9-tetrahydrocannabinol; and
about 0.1-0.4% cannabichromene.

In some embodiments, the ratio of cannabigerol to cannabigerolic acid is selected from the group consisting of about 1:100, about 1:50, about 1:10, and about 1:1. In some embodiments, the ratio of cannabigerol to cannabigerolic acid is about 1:1.

In some embodiments, the concentration of Δ9-tetrahydrocannabinol is insufficient to produce a psychotropic effect. In some embodiments, the ratio of Δ9-tetrahydrocannabinol to the other cannabinoids is about 1:25. In some embodiments, the concentration of Δ9-tetrahydrocannabinol is less than about 10 mg/mL. In some embodiments, the concentration of Δ9-tetrahydrocannabinol is less than about 5 mg/mL. In some embodiments, the concentration of Δ9-tetrahydrocannabinol is less than about 3 mg/mL. In some embodiments, the concentration of Δ9-tetrahydrocannabinol is less than about 2 mg/mL. In some embodiments, the concentration of Δ9-tetrahydrocannabinol is less than about 1 mg/mL. In some embodiments, the concentration of Δ9-tetrahydrocannabinol is less than about 0.5 mg/mL. In some embodiments, the concentration of Δ9-tetrahydrocannabinol is less than about 0.3 mg/mL. In some embodiments, the concentration of Δ9-tetrahydrocannabinol is less than about 0.2 mg/mL. In some embodiments, the concentration of Δ9-tetrahydrocannabinol is less than about 0.1 mg/mL. In some embodiments, the concentration of Δ9-tetrahydrocannabinol is about 0 mg/mL.

In some embodiments, the concentration of Δ9-tetrahydrocannabinol is less than about 1%. In some embodiments, the concentration of Δ9-tetrahydrocannabinol is less than about 0.5%. In some embodiments, the concentration of Δ9-tetrahydrocannabinol is less than about 0.3%. In some embodiments, the concentration of Δ9-tetrahydrocannabinol is less than about 0.2%. In some embodiments, the concentration of Δ9-tetrahydrocannabinol is less than about 0.1%. In some embodiments, the concentration of Δ9-tetrahydrocannabinol is less than about 0.01%.

In some embodiments, the hemp extract comprises:
about 1-10 mg/mL of cannabigerol;
about 1-10 mg/mL of cannabigerolic acid;
less than 0.1 mg/mL Δ9-tetrahydrocannabinol; and
about 0.1-0.4 mg/mL cannabichromene.

In some embodiments, the hemp extract comprises:
about 5 mg/mL of cannabigerol;
about 5 mg/mL of cannabigerolic acid;
less than 0.1 mg/mL Δ9-tetrahydrocannabinol; and
about 0.27 mg/mL cannabichromene.

In some embodiments, the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

In some embodiments, the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

In some embodiments, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In some embodiments, the hemp extract comprises:
about 5% of cannabigerol;
about 5% of cannabigerolic acid;
less than 0.1% Δ9-tetrahydrocannabinol; and
about 0.27% cannabichromene.

In some embodiments, the hemp extract comprises:
α-pinene;
β-myrcene;
β-pinene;
δ-limonene;
linalool;
β-caryophyllene;
α-humulene;
nerolidol;
guaiol;
caryophyllene oxide; and
α-bisabolol.

In some embodiments, the hemp extract comprises
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

In some embodiments, the hemp extract comprises:
camphene;
β-ocimene;
eucalyptol;
isopulegol; and/or
nerolidol.

In some embodiments, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

The disclosure also provides a dosage form comprising any of the pharmaceutical compositions described herein and one or more pharmaceutically acceptable additives, flavoring agents, surfactants, and adjuvants. In some embodiments, the flavoring agent is selected from the group consisting of peppermint oil, mango extract, beef, poultry, and seafood. In some embodiments, the dosage form is formulated as a sublingual spray. In some embodiments, the dosage form is formulated as a water or alcohol soluble solution, or a cream for transdermal application. In some embodiments, the dosage form formulated as a gel for buccal or mucosal administration. In some embodiments, the dosage form is formulated as a powder. In some embodiments, the dosage form is formulated as a solution for subcutaneous injection. In some embodiments, the dosage form is formulated as a tablet. In some embodiments, the dosage form is formulated as a capsule. In some embodiments, the dosage form is formulated as a hard chewable. In some embodiments, the dosage form is formulated as a soft chewable. In some embodiments, the dosage form is formulated for administration using a nebulizer. In some embodiments, the dosage form is formulated for administration using a pet collar.

In some embodiments, the dosage form is formulated as a chew for oral administration. In some embodiments, the chew is produced using cold extrusion. In some embodiments, the weight of the chew is about 0.5-10 g. In some embodiments, the weight of the chew is about 4 g, about 6 g, about 9 g, or about 10 g. In some embodiments, the weight of the chew is about 4 g. In some embodiments, the chew comprises:
about 7 mg of cannabigerol;
about 6 mg of cannabigerolic acid;
about 0.32 mg Δ9-tetrahydrocannabinol; and
about 0.36 mg cannabichromene.

In some embodiments, the dosage form is formulated in a carrier for oral administration. In some embodiments, the carrier is selected from the group consisting of linseed oil, olive oil, fish oil, salmon oil, coconut oil, catnip oil, sesame oil, MCT oil, and grapeseed oil. In some embodiments, the carrier comprises grapeseed oil. In some embodiments, the carrier comprises catnip oil. In some embodiments, the carrier comprises sesame oil.

In some embodiments, the dosage form is formulated for inhalation.

The disclosure also provides a method for treating or reducing pain in a subject in need thereof, comprising administering to the subject a therapeutically effective amount of any of the compositions or dosage forms disclosed herein. In some embodiments, the pain is associated with arthritis, post-operative pain, acute pain, joint pain, or multi-joint pain.

The disclosure also provides a method for treating epilepsy in a subject in need thereof, comprising administering to the subject a therapeutically effective amount of any of the compositions or dosage forms disclosed herein. In some embodiments, the subject has previously experienced generalized motor seizures or focal seizure episodes. In some embodiments, the subject has a decrease in the frequency and/or duration of seizures.

The disclosure also provides a method for improving quality of life in a subject with cancer, comprising administering to the subject a therapeutically effective amount of any of the compositions or dosage forms disclosed herein. In some embodiments, the subject is receiving L-CHOP or CHOP chemotherapy. In some embodiments, the hemp extract, composition or dosage form is administered about every 12 hours starting at week 4 or 5 of doxorubicin treatment. In some embodiments, the cancer is lymphoma. In some embodiments, the lymphoma is intermediate to high-grade multicentric lymphoma. In some embodiments, following treatment the subject experiences an absence of lymphoma-associated abnormalities or a decrease in lymph node diameter. In some embodiments, the subject has a body weight >15 kg. In some embodiments, the subject is entering the end of the first cycle of L-CHOP chemotherapy.

The disclosure also provides a method for treating post-operative pain in a subject in need thereof, comprising administering to the subject a therapeutically effective amount of any of the compositions or dosage forms disclosed herein. In some embodiments, the subject has undergone tibial plateau leveling osteotomy surgery. In some embodiments, the subject has been treated with fentanyl and/or a nerve block.

In some embodiments, any of the pharmaceutical compositions or dosage forms disclosed herein are administered at a dosage of about 0.1-8.0 mg/kg. In some embodiments, any of the pharmaceutical compositions or dosage forms disclosed herein are administered at twice the therapeutically effective dosage for one week, and then subsequently administered at a therapeutically effective dosage. In some embodiments, the therapeutically effective dosage is about 0.1-0.5 mg/kg. In some embodiments, the therapeutically effective dosage is about 2 mg/kg. In some embodiments, the therapeutically effective dosage is about 8 mg/kg.

In some embodiments, any of the pharmaceutical compositions or dosage forms disclosed herein are administered at a dosage of about 1 mg/kg for one week, and then subsequently administered at a dosage of about 0.1-0.5 mg/kg. In some embodiments, any of the pharmaceutical compositions or dosage forms disclosed herein are administered at a dosage of about 4 mg/kg for one week, and then subsequently administered at a dosage of about 2 mg/kg. In some embodiments, any of the methods disclosed herein result in a therapeutically effective median maximal serum concentration of cannabigerol. In some embodiments, the median maximal serum concentration of cannabigerol is about 50 ng/mL. In some embodiments, the median maximal serum concentration of cannabigerolic acid is about 2000 ng/mL.

In some embodiments, the subject is veterinary. In some embodiments, the veterinary subject is canine, feline, bovine, porcine, or equine. In some embodiments, the subject is human.

The disclosure also provides a method of achieving an area under the curve from 0 time to 24 hours of between 42.4 and 3048 ng hr/ml for cannabigerol in a subject comprising administering to the subject an effective amount of hemp extract. In some embodiments, the subject is human, canine or feline.

The disclosure also provides a method of treating or reducing pain in a subject in need thereof, comprising administering a therapeutically effective amount of a pharmaceutical composition comprising hemp extract and a carrier, wherein the hemp extract comprises:
cannabigerol; and
cannabigerolic acid;

wherein the ratio of cannabigerol to cannabigerolic acid is about 0.6:1 to about 1:0.6.

The disclosure also provides a method of treating epilepsy in a subject in need thereof, comprising administering a therapeutically effective amount of a pharmaceutical composition comprising hemp extract and a carrier, wherein the hemp extract comprises:
cannabigerol; and
cannabigerolic acid;

wherein the ratio of cannabigerol to cannabigerolic acid is about 0.6:1 to about 1:0.6.

The disclosure also provides a method of treating cancer in a subject in need thereof, comprising administering a therapeutically effective amount of a pharmaceutical composition comprising hemp extract and a carrier, wherein the hemp extract comprises:
cannabigerol; and
cannabigerolic acid;

wherein the ratio of cannabigerol to cannabigerolic acid is about 0.6:1 to about 1:0.6.

The disclosure also provides a method of improving quality of life in a subject with cancer, comprising administering a therapeutically effective amount of a pharmaceutical composition comprising hemp extract and a carrier, wherein the hemp extract comprises:
cannabigerol; and
cannabigerolic acid;

wherein the ratio of cannabigerol to cannabigerolic acid is about 0.6:1 to about 1:0.6.

In some embodiments, the hemp extract further comprises:
cannabigerolic acid;
Δ9-tetrahydrocannabinol; and
cannabichromene;

In some embodiments, the hemp extract further comprises four or more of the following:
α-pinene;
β-myrcene;
β-pinene;
δ-limonene;
linalool;
β-caryophyllene;
α-humulene;
nerolidol;
guaiol;
caryophyllene oxide; and
α-bisabolol.

In some embodiments, the concentration of Δ9-tetrahydrocannabinol is insufficient to produce a psychotropic effect. In some embodiments the ratio of Δ9-tetrahydrocannabinol to the other cannabinoids is about 1:25. In some embodiments, the concentration of Δ9-tetrahydrocannabinol is less than about 10 mg/mL. In some embodiments, the concentration of Δ9-tetrahydrocannabinol is less than about 5 mg/mL. In some embodiments, the concentration of Δ9-tetrahydrocannabinol is less than about 3 mg/mL. In some embodiments, the concentration of Δ9-tetrahydrocannabinol is less than about 2 mg/mL. In some embodiments, the concentration of Δ9-tetrahydrocannabinol is less than about 1 mg/mL. In some embodiments, the concentration of Δ9-tetrahydrocannabinol is less than about 0.5 mg/mL. In some embodiments, the concentration of Δ9-tetrahydrocannabinol is less than about 0.3 mg/mL. In some embodiments, the concentration of Δ9-tetrahydrocannabinol is less than about 0.2 mg/mL. In some embodiments, the concentration of Δ9-tetrahydrocannabinol is less than about 0.1 mg/mL. In some embodiments, the concentration of Δ9-tetrahydrocannabinol is about 0 mg/mL.

In some embodiments, the concentration of Δ9-tetrahydrocannabinol is less than about 1%. In some embodiments, the concentration of Δ9-tetrahydrocannabinol is less than about 0.5%. In some embodiments, the concentration of Δ9-tetrahydrocannabinol is less than about 0.3%. In some embodiments, the concentration of Δ9-tetrahydrocannabinol is less than about 0.2%. In some embodiments, the concentration of Δ9-tetrahydrocannabinol is less than about 0.1%. In some embodiments, the concentration of Δ9-tetrahydrocannabinol is less than about 0.01%.

In some embodiments, the hemp extract comprises:
about 1-10 mg/mL of cannabigerol;
about 1-10 mg/mL of cannabigerolic acid;
less than 0.1 mg/mL Δ9-tetrahydrocannabinol; and
about 0.1-0.4 mg/mL cannabichromene.

In some embodiments, the hemp extract comprises:
about 5 mg/mL of cannabigerol;
about 5 mg/mL of cannabigerolic acid;
less than 0.1 mg/mL Δ9-tetrahydrocannabinol; and
about 0.27 mg/mL cannabichromene.

In some embodiments, the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

In some embodiments, the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

In some embodiments, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In some embodiments, the hemp extract comprises:
about 5% of cannabigerol;
about 5% of cannabigerolic acid;
less than 0.1% Δ9-tetrahydrocannabinol; and
about 0.27% cannabichromene.

In some embodiments, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

In some embodiments, the hemp extract further comprises:
camphene;
β-ocimene;
eucalyptol;
isopulegol; and/or
nerolidol.

In some embodiments, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

In some embodiments, the hemp extract comprises 5, 6, 7, 8, 9, 10, 11, 12, 13, 14, 15 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In some embodiments, any of the compositions disclosed herein can be formulated in a carrier. In some embodiments, the carrier is selected from the group consisting of linseed oil, olive oil, fish oil, salmon oil, coconut oil, catnip oil, sesame oil, MCT oil, and grapeseed oil. In some embodiments, the carrier comprises grapeseed oil. In some embodiments, the carrier comprises catnip oil. In some embodiments, the carrier comprises sesame oil.

In some embodiments, any of the compositions disclosed herein can comprise nepetalactone. In some embodiments, any of the compositions disclosed herein can comprise taurine.

In some embodiments, any of the compositions disclosed herein can be formulated for administration using a nebulizer. In some embodiments, any of the compositions disclosed herein can be formulated for administration using a diffuser. In some embodiments, any of the compositions disclosed herein can be formulated for administration using a pet collar. In some embodiments, any of the compositions disclosed herein can be formulated as a pet food for oral administration.

In some embodiments, any of the compositions disclosed herein can formulated as a chew for oral administration. In some embodiments, the weight of the chew is about 0.5-10 g. In some embodiments, the weight of the chew is about 4 g, about 6 g, about 9 g, or about 10 g. In some embodiments, the weight of the chew is about 4 g. In some embodiments, the chew comprises:
about 7 mg of cannabigerol;
about 6 mg of cannabigerolic acid;
about 0.32 mg Δ9-tetrahydrocannabinol; and
about 0.36 mg cannabichromene.

In some embodiments, the chew comprises:
about 70 mg of cannabigerol;
about 60 mg of cannabigerolic acid;
about 3.2 mg Δ9-tetrahydrocannabinol; and
about 3.6 mg cannabichromene.

### BRIEF DESCRIPTION OF THE DRAWINGS

Aspects, features, benefits, and advantages of the embodiments described herein will be apparent with regard to the following description, appended claims, and accompanying drawings where:

FIG. 1 shows serum levels of CBG at the indicated time points for fed and fasted dogs treated with a hemp extract composition.

FIG. 2 shows serum levels of CBGA at the indicated time points for fed and fasted dogs treated with a hemp extract composition.

FIG. 3 shows serum levels of CBG after 1 week and 2 weeks of twice daily dosing of dogs with a hemp extract composition.

FIG. 4 shows serum levels of CBGA after 1 week and 2 weeks of twice daily dosing of dogs with a hemp extract composition.

### DETAILED DESCRIPTION

The endocannabinoid receptor system is known to play a role in pain modulation and attenuation of inflammation. Cannabinoid receptors (CB1 and CB2) are widely distributed throughout the central and peripheral nervous system and are also present in the synovium. However, the psychotropic effects of certain cannabinoids prevent extensive research into their use as single agents for pain relief. The cannabinoids are a group of as many as 60 different compounds that may or may not act at CB receptors.

### Epilepsy

The use of cannabinoid derivatives in the treatment of a variety of neurological disorders in humans has recently been explored, particularly in the treatment of chronic pain and epilepsy. Full spectrum cannabinoid rich industrial hemp products below 0.3% THC have been shown to have no psychotropic effects and modest activity through non-cannabinoid receptor routes affecting the serotonergic, glycinergic and GABAergic neurotransmission pathways. Recent research using full spectrum cannabinoid rich HBNs has revealed efficacy of these products in dogs with chronic pain (Wakshlag et al., Front Vet Sci, 2018). Additionally, investigation in epilepsy in humans and the release of Epidiolex as viable treatment shows merits to cannabidiol in the treatment of epilepsy, but there have been no published investigations evaluating efficacy of HBNs in a canine epilepsy model. There is a need to evaluate whether treatment of refractory canine epilepsy with a HBN will decrease seizure numbers or duration and whether it will alter metabolism of other commonly used drugs for seizure control in dogs.

### Post-Operative Pain Relief

The use of cannabinoid derivatives in the treatment of a variety of neurological disorders in humans has recently been explored, particularly in the treatment of chronic pain and epilepsy. Full spectrum cannabinoid rich Industrial hemp based nutraceuticals (HBN) below 0.3% THC have been shown to have no psychotropic effects and modest activity through non-cannabinoid receptor routes affecting the serotonergic, glycinergic and GABA neurotransmission pathways that may be able to diminish pain, as well as inflammation. Recent research into chronic pain has revealed efficacy of these. HBN in diminishing chronic osteoarthritic pain based on objective client-based validated surveys, however post-surgical pain relief is still unexamined. The use of a HBN should help alleviate post-operative pain and improve outcomes in dogs undergoing tibial plateau leveling osteotomy surgery for cranial cruciate rupture.

In addition, cannabinoids may be useful in alleviating post-operative pain following spinal cord injury and hemilaminectomy. Post-operative pain following spinal cord injury and hemilaminectomy is complex and involves inflammatory nociceptive and neuropathic mechanisms of pain. The complexity of post-operative hemilaminectomy pain necessitates a multimodal analgesia protocol. Current standard of care in post-operative hemilaminectomy patients is parenteral opioids with protocols for adjunctive analgesics varying by institution. While effective in controlling post-operative pain, opioid use can also be associated with undesirable adverse effects such as: vomiting, inappetence, dysphoria, central nervous system and respiratory depression, constipation and sedation. The goal of adjunctive analgesia in post-operative hemilaminectomy patients is two-fold, to decrease the amount of opioids needed for pain control and to target multiple mechanisms of pain. Typical adjunctive analgesic medications may include gabapentin, diazepam, and non-steroidal anti-inflammatory or steroid medications. Gabapentin, a gold-standard for neuropathic pain, is generally well tolerated by patients and is commonly used in post-operative neurosurgery patients.

The present disclosure is directed toward compositions comprising hemp extract and their use for the treatment of pain in animals. Also provided herein are methods for treatment of pain in veterinary subjects. The efficacy of these compositions and treatment methods has not previously been demonstrated. Clinical trial and pharmacokinetic data regarding dosing is also provided herein.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs.

Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±5%, from the specified value, as such variations are appropriate to perform the disclosed methods.

As used in the specification and in the claims, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “treatment” or “treating,” is defined as the application or administration of a therapeutic agent, i.e., a compound provided herein (alone or in combination with another pharmaceutical agent), to a patient, or application or administration of a therapeutic agent to an isolated tissue or cell line from a patient (e.g., for diagnosis or ex vivo applications), with the purpose to cure, heal, alleviate, relieve, alter, remedy, ameliorate, improve or affect the symptoms of a disease, disorder, syndrome, or condition. Such treatments can be specifically tailored or modified, based on knowledge obtained from the field of pharmacogenomics.

In certain embodiments, the compositions described herein reduce pain in a subject. Pain can be measured using any metric known in the art. For example, pain can be measured using the canine brief pain inventory (CBPI), the Hudson activity scale, flexion and tension measurements and gait analysis. A reduction in any of these metrics shows a treatment of or reduction in pain.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “use” includes any one or more of the following embodiments of the invention, respectively: the use in the treatment of pain the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise.

As used herein, the term “patient,” “individual,” or “subject” is intended to include organisms, e.g., prokaryotes and eukaryotes, which are capable of suffering from or afflicted with a disease, disorder or condition associated with the activity of a protein kinase. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from, schizophrenia. In another embodiment, the subject is a cell.

When used with respect to methods of treatment/prevention and the use of the compounds and pharmaceutical compositions thereof described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.). Typically, when a step of administering a compound of the invention is disclosed herein, the invention further contemplates a step of identifying an individual or subject in need of the particular treatment to be administered or having the particular condition to be treated.

In some embodiments, the individual is a mammal, including, but not limited to, bovine, equine, feline, rabbit, canine, rodent, or primate. In some embodiments, the mammal is a primate. In some embodiments, the primate is a human. In some embodiments, the individual is human, including adults, children and premature infants. In some embodiments, the individual is a non-mammal. In some variations, the primate is a non-human primate such as chimpanzees and other apes and monkey species. The term “individual” does not denote a particular age or sex.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” or “carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; tale; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds can also be incorporated into the compositions. The “pharmaceutically acceptable carrier” or “cater” can further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “stabilizer,” as used herein, refers to polymers capable of chemically inhibiting or preventing degradation. Stabilizers are added to formulations of compounds to improve chemical and physical stability of the compound.

As used herein, the term “adjuvant” may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “weight percent” is meant to refer to the quantity by weight of a compound and/or component in a composition as the quantity by weight of a constituent component of the composition as a percentage of the weight of the total composition. The weight percent can also be calculated by multiplying the mass fraction by 100. The “mass fraction” is the ratio of one substance of a mass ml to the mass of the total composition m_{T} such that weight percent=(m₁/m_{T})*100. Wherever “%” is used herein, it is assumed to be weight percent unless labeled otherwise.

“Aqueous buffer” refers to a water solution which resists change in hydronium ion and the hydroxide ion concentration (and consequent pH) upon addition of small amounts of acid or base, or upon dilution. Buffer solutions consist of a weak acid and its conjugate base (more common) or a weak base and its conjugate acid (less common). The buffer can be prepared by methods well known in the art with the appropriate buffering agents to give the desired pH value. Examples of the suitable buffering agents include hydrochloric acid, lactic acid, acetic acid, citric acid, malic acid, maleic acid, pyruvic acid, succinic acid, tris-hydroxymethylaminomethane, sodium hydroxide, sodium bicarbonate, phosphoric acid, sodium phosphate, and other biologically acceptable buffering agents. Aqueous buffers are readily available commercially and they can be used in preparation of the compositions of this invention without further treatment.

As used herein, the term “hemp extract” refers to a composition of cannabinoids and terpenes that are isolated from a hemp plant. The terms “hemp extract” and “CBG/CBGA oil” have the same meaning and are used interchangeably herein. The hemp extract can be obtained by any method known in the art. For example, the hemp extract can be obtained by supercritical (or subcritical) CO₂ extraction, which uses carbon dioxide under high pressure and low temperatures to isolate, preserve and maintain the purity of hemp extract. In an embodiment, the hemp extract is obtained from a supercritical CO₂ extraction. For example, supercritical CO₂ extraction may be performed as described in U.S. Pat. No. 8,895,078, which is incorporated herein by reference in its entirety. Alternatively, a solvent such as petroleum ether, ethanol, methanol, butanol, acetone, dry ice, or olive oil can be used, at room temperature (ambient temperature) with stirring, by passive extraction, heated to a temperature above room temperature, or under reflux, as known in the art to provide the hemp extract. In another embodiment, hemp extract from a butanol extraction is employed as starting material for methods disclosed herein. In some embodiments, the hemp extract undergoes additional steps to concentrate one or more components of the hemp extract. In some embodiments, the hemp extract is enriched for the presence of CBG and/or CBGA.

Suitable methods for measuring the cannabinoid and terpene content in the hemp extract are known in the art. In an embodiment, cannabinoid content is determined using liquid chromatography with mass spectrometry detection (LC-MS). In another embodiment, terpene content is determined using gas chromatography with flame ionization detection (GC-FID) analysis of headspace.

As used herein, the term “flavoring agent” refers to an ingredient that is added to a composition to impart a particular flavor, smell, or other organoleptic property.

As used herein, the term “oil” refers to a nonpolar viscous liquid that is both hydrophobic and lipophilic. Oils may be isolated from animal, vegetable, or petrochemical products.

As used herein, the term “chew” refers to a product or a portion thereof that has rheological and other texture and organoleptic properties which tend to promote chewing upon the article by a target animal. Generally speaking, a chewable matrix will exhibit sufficient ductility that it is at least slightly malleable when bitten by the target animal and sufficient palatability that the target animal is not deterred by its taste from biting it multiple times. By contrast, “chewable” does not mean merely that an article can be chewed by an animal (i.e., it does not mean merely that some portion of the article will fit within an animal's mouth sufficiently to permit engagement of the animal's teeth against the portion).

The “maximal serum concentration level” of a substance, as used herein, refers to the maximal level of the substance found in a plasma sample following a single administration.

As used herein, the term “cold extrusion” refers to a process for producing edible food products comprising several unit operations including mixing, kneading, shearing, shaping, and forming, all of which are conducted at or near ambient temperature.

As used herein, the term “psychotropic effect” refers to a modification of brain function that results in an alteration of perception, mood, consciousness, or behavior.

As used herein, “chemotherapy” is any chemical compound used in the treatment of a proliferative disorder. Examples of chemotherapeutic agents include, without being limited to, the following classes of agents:
nitrogen mustards, e.g. cyclophosphamide, trofosfamide, ifosfamide and chlorambucil; nitroso ureas, e.g. carmustine (BCNU), lomustine (CCNU), semustine (methyl CCNU) and nimustine (ACNU);
ethylene imines and methyl-melamines, e.g. thiotepa;
folic acid analogs, e.g. methotrexate;
pyrimidine analogs, e.g. 5-fluorouracil and cytarabine;
purine analogs, e.g. mercaptopurine and azathioprine;
vinca alkaloids, e.g. vinblastine, vincristine and vindesine;
epipodophyllotoxins, e.g. etoposide and teniposide;
antibiotics, e.g. dactinomycin, daunorubicin, doxorubicin, epirubicin, bleomycin a2, mitomycin c and mitoxantrone;
estrogens, e.g. eiethyl stilbestrol;
gonadotropin-releasing hormone analogs, e.g. leuprolide, buserelin and goserelin;
antiestrogens, e.g. tamoxifen and aminoglutethimide;
androgens, e.g. testolactone and drostanolonproprionate;
platinates, e.g. cisplatin and carboplatin; and interferons, including interferon-alpha, beta and gamma.

As used herein, the term “quality of life”, or “QoL,” is generally considered a multidimensional concept that involves subjective evaluation of factors that contribute to overall well-being with a more recent publication suggesting a malaise, anxiety and digestive function. Likert scaling system appears to be a sound assessment of QoL (Giuffrida et al. (2018) J Amer Vet Med Assoc. 252:1073-1083.) and is used in this study.

### Pharmaceutical Compositions

In an aspect, provided herein is a pharmaceutical composition comprising hemp extract and a carrier, wherein the hemp extract comprises:
cannabigerol; and
cannabigerolic acid.

In another embodiment, the hemp extract comprises:
cannabigerol;
cannabigerolic acid;
cannabidiol;
cannabidiolic acid;
Δ9-tetrahydrocannabinol; and
cannabichromene.

In another embodiment, the ratio of Δ9-tetrahydrocannabinol to the other cannabinoids is from about 1:50 to about 1:20. In an embodiment, the ratio of cannabigerol to cannabigerolic acid is about 0.1:1 to about 1:0.1. In another embodiment, the ratio of cannabigerol to cannabigerolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabigerol to cannabigerolic acid is about 0.2:1 to about 1:0.2. In yet another embodiment, the ratio of cannabigerol to cannabigerolic acid is about 0.4:1 to about 1:0.4. In yet another embodiment, the ratio of cannabigerol to cannabigerolic acid is about 0.6:1 to about 1:0.6. In still another embodiment, the ratio of cannabigerol to cannabigerolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 20 mg/mL. In another embodiment, the concentration of Δ9-tetrahydrocannabinol is less than about 10 mg/mL. In another embodiment, the concentration of Δ9-tetrahydrocannabinol is less than about 5 mg/mL. In another embodiment, the concentration of Δ9-tetrahydrocannabinol is less than about 3 mg/mL. In another embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is about 0 mg/mL.

In some embodiments, the concentration of Δ9-tetrahydrocannabinol is less than about 2%. In some embodiments, the concentration of Δ9-tetrahydrocannabinol is less than about 1%. In some embodiments, the concentration of Δ9-tetrahydrocannabinol is less than about 0.5%. In some embodiments, the concentration of Δ9-tetrahydrocannabinol is less than about 0.3%. In some embodiments, the concentration of Δ9-tetrahydrocannabinol is less than about 0.2%. In some embodiments, the concentration of Δ9-tetrahydrocannabinol is less than about 0.1%. In some embodiments, the concentration of Δ9-tetrahydrocannabinol is less than about 0.01%.

In an embodiment, the hemp extract comprises:
about 0.1-20 mg/mL of cannabigerol;
about 0.1-20 mg/mL of cannabigerolic acid;
about 0.01-0.5 mg/mL Δ9-tetrahydrocannabinol; and
about 0.01-0.5 mg/mL cannabichromene.

In another embodiment, the hemp extract comprises:
about 1-10 mg/mL of cannabigerol;
about 1-10 mg/mL of cannabigerolic acid;
less than 0.1 mg/mL Δ9-tetrahydrocannabinol; and
about 0.1-0.4 mg/mL cannabichromene.

In yet another embodiment, the hemp extract comprises:
about 5 mg/mL of cannabigerol;
about 5 mg/mL of cannabigerolic acid;
less than 0.1 mg/mL Δ9-tetrahydrocannabinol; and
about 0.27 mg/mL cannabichromene.

In another embodiment, the hemp extract comprises:
about 1-10 mg/mL of cannabigerolic acid;
about 1-10 mg/mL of cannabigerol;
about 0.1-0.5 mg/mL cannabidiol;
about 0.1-0.5 mg/mL cannabidiolic acid;
less than 0.1 mg/mL Δ9-tetrahydrocannabinol; and
about 0.1-0.4 mg/mL cannabichromene.

In another embodiment, the hemp extract comprises:
about 1-200 mg/mL of cannabigerol;
about 1-200 mg/mL of cannabigerolic acid;
less than 0.1-5 mg/mL Δ9-tetrahydrocannabinol; and
about 0.1-5 mg/mL cannabichromene.

In another embodiment, the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

In another embodiment, the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

In another embodiment, the hemp extract comprises:
about 10-100 mg/mL of cannabigerol;
about 10-100 mg/mL of cannabigerolic acid;
about 1-5 mg/mL cannabidiol;
about 1-5 mg/mL cannabidiolic acid;
less than 1 mg/mL Δ9-tetrahydrocannabinol; and
about 1-4 mg/mL cannabichromene.

In another embodiment, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In another embodiment, the hemp extract comprises:
about 5% of cannabigerol;
about 5% of cannabigerolic acid;
less than 0.1% Δ9-tetrahydrocannabinol; and
about 0.27% cannabichromene.

In another embodiment, the hemp extract comprises:
about 0.1-20% of cannabigerol;
about 0.1-20% of cannabigerolic acid;
about 0.01-0.5% Δ9-tetrahydrocannabinol; and
about 0.01-0.5% cannabichromene.

In another embodiment, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In yet another embodiment, the hemp extract comprises:
about 5% of cannabigerol;
about 5% of cannabigerolic acid;
less than 0.1% Δ9-tetrahydrocannabinol; and
about 0.27% cannabichromene.

In another embodiment, the hemp extract comprises:
about 1-10% of cannabigerolic acid;
about 1-10% of cannabigerol;
about 0.1-0.5% cannabidiol;
about 0.1-0.5% cannabidiolic acid;
less than 0.1% Δ9-tetrahydrocannabinol; and
about 0.1-0.4% cannabichromene.

In an embodiment, provided herein is a pharmaceutical composition comprising hemp extract and a carrier, wherein the hemp extract comprises:
α-pinene;
β-myrcene;
β-pinene;
δ-limonene;
linalool;
β-caryophyllene;
α-humulene;
nerolidol;
guaiol;
caryophyllene oxide; and
α-bisabolol.

In another embodiment, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

In another embodiment, the hemp extract comprises:
about 0.07-0.30% α-pinene;
about 0.10-0.60% β-myrcene;
about 0.02-0.20% β-pinene;
about 0.03-0.20% δ-limonene;
about 0.01-0.08% linalool;
about 0.03-0.09% β-caryophyllene;
about 0.01-0.06% α-humulene;
about 0.02-0.09% nerolidol; and
about 0.01-0.06% guaiol;

In another embodiment, the hemp extract comprises:
about 0.01-0.50% α-pinene;
about 0.01-0.90% β-myrcene;
about 0.01-0.50% β-pinene;
about 0.01-0.50% δ-limonene:
about 0.01-0.50% linalool;
about 0.01-0.50% β-caryophyllene;
about 0.01-0.50% α-humulene;
about 0.01-0.50% nerolidol;
about 0.01-0.50% guaiol;
about 0.01-0.50% caryophyllene oxide; and
about 0.01-0.50% α-bisabolol.

In another embodiment, the hemp extract further comprises:
camphene;
β-ocimene;
eucalyptol;
isopulegol; and/or
nerolidol.

In another embodiment, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

In another embodiment, the hemp extract comprises:
about 0.01-0.04% camphene;
about 0.01-0.05% β-ocimene;
about 0.01-0.07% eucalyptol;
about 0.01-0.04% isopulegol; and/or
about 0.01-0.05% nerolidol.

In another embodiment, the hemp extract comprises:
about 0.01-0.50% camphene;
about 0.01-0.50% β-ocimene;
about 0.01-0.50% eucalyptol;
about 0.01-0.50% isopulegol; and/or
about 0.01-0.50% nerolidol 1.

In an embodiment, the hemp extract does not comprise terpenes.

In an embodiment, the hemp extract comprises 1 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 2 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 3 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 4 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 5 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 6 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 7 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 8 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 9 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 10 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 11 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 12 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 13 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 14 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises 15 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the hemp extract comprises the following: α-pinene, β-myrcene, β-pinene, 6-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the composition is formulated with a carrier, e.g., with an oil. In some embodiments, the oil acts as a carrier). In another embodiment, the carrier is selected from the group consisting of linseed oil, olive oil, fish oil, salmon oil, coconut oil, catnip oil, sesame oil, MCT oil, and grapeseed oil. In yet another embodiment, the carrier comprises grapeseed oil. In yet another embodiment, the carrier comprises sesame oil.

In an embodiment, the pharmaceutical composition comprises nepetalactone.

In an embodiment, the pharmaceutical composition comprises taurine.

In an embodiment, the pharmaceutical composition comprises lecithin. In another embodiment, the lecithin is sunflower lecithin. In another embodiment. the lecithin is about 5%, about 10%, about 15%, about 20%, about 25%, about 30%, about 35%, about 40%, about 45%, or 50% w/v of the pharmaceutical composition. In other embodiments, the pharmaceutical composition comprises lecithin mixed with another oil. In some embodiments, the other oil is sesame oil. In some embodiments, the lecithin and other oil are mixed at a ratio of about 1:4, 1:2, 1:1, 2:1 or 4:1. In some embodiments, the pharmaceutical composition comprises hemp extract and a carrier oil. In some embodiments, the carrier oil is substantially all lecithin. In other embodiments, the carrier oil is 100% lecithin.

In an embodiment, the pharmaceutical composition comprises NF-971P. In an embodiment, the NF-971P is about 0.5%, about 1.0%, about 1.5%, about 2.0%, about 2.5%, or about 3.0% weight/volume ratio of the pharmaceutical composition.

In an embodiment, the pharmaceutical composition is formulated as a sublingual spray. In still another embodiment, the pharmaceutical composition is formulated as a water or alcohol soluble solution, a gel, or a cream for transdermal application. In an embodiment, the pharmaceutical composition is formulated as a gel for buccal or mucosal administration. In an embodiment, the pharmaceutical composition is formulated as a powder. In another embodiment, the pharmaceutical composition is formulated as a solution for subcutaneous injection. In yet another embodiment, the pharmaceutical composition is formulated as a tablet. In still another embodiment, the pharmaceutical composition is formulated as a capsule. In an embodiment, the pharmaceutical composition is formulated as a hard chewable. In an embodiment, the pharmaceutical composition is formulated as a soft chewable.

In an embodiment, the composition is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In an embodiment, the 4 g chew comprises:
about 7 mg of cannabigerol;
about 6 mg of cannabigerolic acid;
about 0.32 mg Δ9-tetrahydrocannabinol; and
about 0.36 mg cannabichromene.

In an embodiment, the 4 g chew comprises:
about 70 mg of cannabigerol;
about 60 mg of cannabigerolic acid;
about 3.2 mg Δ9-tetrahydrocannabinol; and
about 3.6 mg cannabichromene.

The pharmaceutical compositions of the present disclosure may be manufactured by processes well known in the art, e.g., by means of conventional mixing, dissolving, granulating, grinding, pulverizing, dragee-making, levitating, emulsifying, encapsulating, entrapping or by lyophilizing processes.

The compositions for use in accordance with the present disclosure thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

### Dosage Forms

In an aspect, provided herein is a dosage form comprising:
cannabigerol; and
cannabigerolic acid; and
one or more pharmaceutically acceptable additives, flavoring agents, surfactants, and adjuvants.

In another embodiment, the dosage form comprises:
cannabigerol;
cannabigerolic acid;
cannabidiol;
cannabidiolic acid;
Δ9-tetrahydrocannabinol;
cannabichromene; and
one or more pharmaceutically acceptable additives, flavoring agents, surfactants, and adjuvants.

In an embodiment, the ratio of cannabigerol to cannabigerolic acid is selected from the group consisting of about 1:100, about 1:50, about 1:10, and about 1:1. In an embodiment, the ratio of cannabigerol to cannabigerolic acid is about 0.1:1 to about 1:0.1. In another embodiment, the ratio of cannabigerol to cannabigerolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabigerol to cannabigerolic acid is about 0.6:1 to about 1:0.6. In another embodiment, the ratio of cannabigerol to cannabigerolic acid is about 0.2:1 to about 1:0.2. In yet another embodiment, the ratio of cannabigerol to cannabigerolic acid is about 0.4:1 to about 1:0.4. In still another embodiment, the ratio of cannabigerol to cannabigerolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment; the ratio of Δ9-tetrahydrocannabinol to the other cannabinoids is about 1:50. In still another embodiment, the ratio of Δ9-tetrahydrocannabinol to the other cannabinoids is about 1:45. In an embodiment; the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 20 mg/mL. In another embodiment, the concentration of Δ9-tetrahydrocannabinol is less than about 10 mg/mL. In another embodiment, the concentration of Δ9-tetrahydrocannabinol is less than about 5 mg/mL. In another embodiment, the concentration of Δ9-tetrahydrocannabinol is less than about 3 mg/mL. In another In another embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment; the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment. the concentration of Δ9-tetrahydrocannabinol is about 0 mg/mL.

In some embodiments, the concentration of Δ9-tetrahydrocannabinol is less than about 2%. In some embodiments, the concentration of Δ9-tetrahydrocannabinol is less than about 1%. In some embodiments, the concentration of Δ9-tetrahydrocannabinol is less than about 0.5%. In some embodiments, the concentration of Δ9-tetrahydrocannabinol is less than about 0.3%. In some embodiments, the concentration of Δ9-tetrahydrocannabinol is less than about 0.2%. In some embodiments, the concentration of Δ9-tetrahydrocannabinol is less than about 0.1%. In some embodiments, the concentration of Δ9-tetrahydrocannabinol is less than about 0.01%.

In an embodiment, the dosage form comprises:
about 0.1-20 mg/mL of cannabigerol;
about 0.1-20 mg/mL of cannabigerolic acid;
about 0.01-0.5 mg/mL Δ9-tetrahydrocannabinol; and
about 0.01-0.5 mg/mL cannabichromene.

In another embodiment, the dosage form comprises:
about 1-10 mg/mL of cannabigerol;
about 1-10 mg/mL of cannabigerolic acid;
less than 0.1 mg/mL Δ9-tetrahydrocannabinol; and
about 0.1-0.4 mg/mL cannabichromene.

In yet another embodiment, the dosage form comprises:
about 5 mg/mL of cannabigerol;
about 5 mg/mL of cannabigerolic acid;
less than 0.1 mg/mL Δ9-tetrahydrocannabinol; and
about 0.27 mg/mL cannabichromene.

In another embodiment, the hemp extract comprises:
about 1-200 mg/mL of cannabigerol;
about 1-200 mg/mL of cannabigerolic acid;
less than 0.1-5 mg/mL Δ9-tetrahydrocannabinol; and
about 0.1-5 mg/mL cannabichromene.

In another embodiment, the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

In another embodiment, the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

In another embodiment, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In another embodiment, the hemp extract comprises:
about 5% of cannabigerol;
about 5% of cannabigerolic acid;
less than 0.1% Δ9-tetrahydrocannabinol; and
about 0.27% cannabichromene.

In an embodiment, the hemp extract comprises:
about 0.1-20% of cannabigerol;
about 0.1-20% of cannabigerolic acid;
about 0.01-0.5% Δ9-tetrahydrocannabinol; and
about 0.01-0.5% cannabichromene.

In another embodiment, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In yet another embodiment, the hemp extract comprises:
about 5% of cannabigerol;
about 5% of cannabigerolic acid;
less than 0.1% Δ9-tetrahydrocannabinol; and
about 0.27% cannabichromene.

In some embodiments, the dosage form comprises:
α-pinene;
β-myrcene;
β-pinene;
δ-limonene;
linalool;
β-caryophyllene;
α-humulene;
nerolidol;
guaiol;
caryophyllene oxide; and
α-bisabolol.

In another embodiment, the dosage form comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

In another embodiment, the dosage form comprises:
about 0.07-0.30% α-pinene;
about 0.10-0.60% β-myrcene;
about 0.02-0.20% β-pinene;
about 0.03-0.20% δ-limonene;
about 0.01-0.08% linalool;
about 0.03-0.09% β-caryophyllene;
about 0.01-0.06% α-humulene;
about 0.02-0.09% nerolidol; and
about 0.01-0.06% guaiol;

In another embodiment, the dosage form comprises:
about 0.01-0.50% α-pinene;
about 0.01-0.90% β-myrcene;
about 0.01-0.50% β-pinene;
about 0.01-0.50% δ-limonene;
about 0.01-0.50% linalool;
about 0.01-0.50% β-caryophyllene;
about 0.01-0.50% α-humulene;
about 0.01-0.50% nerolidol;
about 0.01-0.50% guaiol;
about 0.01-0.50% caryophyllene oxide; and
about 0.01-0.50% α-bisabolol.

In another embodiment, the dosage form further comprises:
camphene;
β-ocimene;
eucalyptol;
isopulegol; and/or
nerolidol.

In another embodiment, the dosage form comprises:
about 0.02% camphene;
about 0.02-0.03% β-ocimene;
about 0.02-0.05% eucalyptol;
about 0.02% isopulegol; and/or
about 0.02-0.04% nerolidol.

In another embodiment, the dosage form comprises:
about 0.01-0.04% camphene;
about 0.01-0.05% β-ocimene;
about 0.01-0.07% eucalyptol;
about 0.01-0.04% isopulegol; and/or
about 0.01-0.05% nerolidol.

In another embodiment, the dosage form comprises:
about 0.01-0.50% camphene;
about 0.01-0.50% β-ocimene;
about 0.01-0.50% eucalyptol;
about 0.01-0.50% isopulegol; and/or
about 0.01-0.50% nerolidol.

In an embodiment, the dosage form does not comprise terpenes.

In an embodiment, the dosage form comprises 1 or more of the following: α-pinene, β-myrcene, β-pinene, δ-limonene, linalool, β-caryophyllene. α-humulene, nerolidol, guaiol, caryophyllene oxide, α-bisabolol; camphene, β-ocimene, eucalyptol, isopulegol, and nerolidol.

In an embodiment, the dosage form comprises 2 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 3 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 4 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 5 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 6 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 7 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 8 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 9 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 10 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 11 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 12 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 13 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 14 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises 15 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

In an embodiment, the dosage form comprises the following: α-pinene, β-myrcene, β-pinene, 6-limonene, linalool, β-caryophyllene, α-humulene, nerolidol, guaiol, caryophyllene oxide, α-bisabolol, camphene, β-ocimene, eucalyptol, isopulegol, and nerolidol

In an embodiment, the flavoring agent is selected from the group consisting of catnip oil, peppermint oil, mango extract, beef, poultry, and seafood.

In an embodiment, the dosage form is formulated as a sublingual spray. In still another embodiment, the dosage form is formulated as a water or alcohol soluble solution, a gel, or a cream for transdermal application. In an embodiment, the dosage form is formulated as a powder. In an embodiment, the dosage form is formulated as a gel for buccal or mucosal administration. In another embodiment, the dosage form is formulated as a solution for subcutaneous injection. In yet another embodiment, the dosage form is formulated as a tablet. In still another embodiment, the dosage form is formulated as a capsule. In an embodiment, the dosage form is formulated as a hard chewable. In an embodiment, the dosage form is formulated as a soft chewable.

In some embodiments, the invention includes infusing edible products with hemp extract. In another embodiment, the edible product is an extruded food product, baked food product, nut butter, spread, pelleted feed, or processed food. In another embodiment, the edible product is a pet food. In another embodiment the pet food is in a dry, shelf-stable form such as dried meals, dried fish, dried dairy products, fish meal, fish flour, cereals, flours, carbohydrates, dried fruits, etc. In another embodiment, the pet food is moist or semi-moist. In another embodiment, the pet food contains food additives or supplements such as vitamins, minerals, medicinals, etc., for example chemicals, enzymes, etc., capable of removing plaque or tartar from the animal's teeth, etc.

In an embodiment, the hemp extract is administered with catnip oil. In another embodiment, any of the dosage forms described can also include catnip.

In another embodiment, hemp extracts are administered using a nebulizer. In another embodiment, the nebulizer delivery device and system is capable of effectively and efficiently administering one or more nebulized drug to an animal. In another embodiment, the nebulizer system can easily be used on animals without removing them from their natural environment. In another embodiment, the nebulizer delivery device and system enables animals to be easily treated daily or multiple times a day without undue stress or the need for extensive resources. In another embodiment, the nebulizer delivery device and system can be used on animals having varying levels of training.

In one embodiment, hemp extract is administered using a diffuser. The diffuser can be any device which disperses hemp extract into the air. Hemp extract may be dispersed by any method, including by natural convection, by forced convection, by heating a wick or pad, for example, holding the hemp extract, by using pumps, or with fans.

In one embodiment, hemp extract is administered by a pet collar. The pet collar may comprise a belt with a buckle on one side, a free end on the other side and an attachment means, such as apertures disposed longitudinally within the central portion of the belt, or a quick release clasp mechanism, for securing the collar in a closed loop configuration. The pet collar may be made from a variety of materials including nylon, polyester leather or other suitable material. The belt material may be treated with a water-proofing compound. The nylon or polyester belt may be interwoven with reflective fibers to enhance the visibility of the pet collar during nighttime hours. In one embodiment, the collar is infused with hemp extract.

### Chews

In an embodiment, the dosage form is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In one embodiment, the dosage form comprises:
brewer's yeast;
arabic gum;
guar gum;
a flavoring agent;
Verdilox;
Previon;
hemp extract;
glycerin;
sunflower lecithin; and
water.

In another embodiment, the dosage form comprises:
about 25-35% brewer's yeast;
about 1-10% arabic gum;
about 0.1-4% guar gum;
about 10-20% of a flavoring agent;
about 0.01-1% Verdilox;
about 0.1-2% Previon;
about 1-10% hemp extract;
about 10-20% glycerin;
about 1-10% sunflower lecithin; and
about 1-10% water.

In another embodiment, the dosage form comprises:
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In yet another embodiment, the dosage form comprises:
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and
about 5.7% water.

In one embodiment, the dosage form comprises:
glucosamine HCl;
brewer's yeast;
arabic gum;
guar gum;
a flavoring agent;
Verdilox;
Previon;
hemp extract;
glycerin;
sunflower lecithin; and
water.

In another embodiment, the dosage form comprises:
about 10-20% glucosamine HCl;
about 25-35% brewer's yeast;
about 1-10% arabic gum;
about 0.1-4% guar gum;
about 10-20% of a flavoring agent;
about 0.01-1% Verdilox;
about 0.1-2% Previon;
about 1-10% hemp extract;
about 10-20% glycerin;
about 1-10% sunflower lecithin; and
about 1-10% water.

In another embodiment, the dosage form comprises:
about 12-17% glucosamine HCl;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In yet another embodiment, the dosage form comprises:
about 15.6% glucosamine HCl;
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and
about 5.7% water.

In one embodiment, the dosage form comprises:
glucosamine HCl;
chondroitin sulfate (76%);
brewer's yeast;
arabic gum;
guar gum;
a flavoring agent;
Verdilox;
Previon;
hemp extract;
glycerin;
sunflower lecithin; and
water.

In another embodiment, the dosage form comprises:
about 10-20% glucosamine HCl;
about 0.1-7% chondroitin sulfate (76%);
about 25-35% brewer's yeast;
about 1-10% arabic gum;
about 0.1-4% guar gum;
about 10-20% of a flavoring agent;
about 0.01-1% Verdilox;
about 0.1-2% Previon;
about 1-10% hemp extract;
about 10-20% glycerin;
about 1-10% sunflower lecithin; and
about 1-10% water.

In another embodiment, the dosage form comprises:
about 12-17% glucosamine HCl;
about 1-4% chondroitin sulfate (76%);
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In yet another embodiment, the dosage form comprises:
about 15.6% glucosamine HCl;
about 2.6% chondroitin sulfate (76%);
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and
about 5.7% water.

In another embodiment, the dosage form comprises:
hyaluronic acid;
brewer's yeast;
arabic gum;
guar gum;
a flavoring agent;
Verdilox;
Previon;
hemp extract;
glycerin;
sunflower lecithin; and
water.

In another embodiment, the dosage form comprises:
about 0.01-3% hyaluronic acid;
about 25-35% brewer's yeast;
about 1-10% arabic gum;
about 0.1-5% guar gum;
about 10-20% of a flavoring agent;
about 0.01-1% Verdilox;
about 0.1-3% Previon;
about 1-10% hemp extract;
about 10-20% glycerin;
about 1-10% sunflower lecithin; and
about 1-10% water.

In another embodiment, the dosage form comprises:
about 0.01-1% hyaluronic acid;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In yet another embodiment, the dosage form comprises:
about 0.1% hyaluronic acid;
about 30.6% brewer's yeast;
about 4.8% arabic gum;
about 0.97% guar gum;
about 14.5% of a flavoring agent;
about 0.05% Verdilox;
about 0.97% Previon;
about 4.8% hemp extract;
about 15.5% glycerin;
about 5.8% sunflower lecithin; and
about 5.8% water.

In another embodiment, the dosage form comprises:
glucosamine HCl;
hyaluronic acid;
brewer's yeast;
arabic gum;
guar gum;
a flavoring agent;
Verdilox;
Previon;
hemp extract;
glycerin;
sunflower lecithin; and
water.

In another embodiment, the dosage form comprises:
about 10-20% glucosamine HCl;
about 0.01-3% hyaluronic acid;
about 25-35% brewer's yeast;
about 1-10% arabic gum;
about 0.1-5% guar gum;
about 10-20% of a flavoring agent;
about 0.01-1% Verdilox;
about 0.1-3% Previon;
about 1-10% hemp extract;
about 10-20% glycerin;
about 1-10% sunflower lecithin; and
about 1-10% water.

In another embodiment, the dosage form comprises:
about 12-17% glucosamine HCl;
about 0.01-1% hyaluronic acid;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In yet another embodiment, the dosage form comprises:
about 16% glucosamine HCl;
about 0.1% hyaluronic acid;
about 30.6% brewer's yeast;
about 4.8% arabic gum;
about 0.97% guar gum;
about 14.5% of a flavoring agent;
about 0.05% Verdilox;
about 0.97% Previon;
about 4.8% hemp extract;
about 15.5% glycerin;
about 5.8% sunflower lecithin; and
about 5.8% water.

In yet another embodiment, the dosage form comprises:
hemp extract;
peanut butter;
rice bran;
glucosamine HCl;
sweet potato;
dry molasses;
sorbic acid
brewer's yeast;
sugar;
water;
glycerin;
potato starch;
dehydrated peanut butter;
rice starch; and
guar gum.

In yet another embodiment, the dosage form comprises:
about 5.0% hemp extract;
about 15.0% peanut butter;
about 12.5% rice bran;
about 5.5% sweet potato;
about 8.0% dry molasses;
about 1% sorbic acid;
about 5.0% brewer's yeast;
about 6.0% sugar;
about 9.25% water;
about 13.0 glycerin;
about 2.0% potato starch;
about 1.0% dehydrated peanut butter;
about 2.0% rice starch; and
about 2.0% guar gum.

In yet another embodiment, the dosage form comprises:
about 5.0% hemp extract;
about 15.0% peanut butter;
about 13.0% rice bran;
about 6.0% sweet potato;
about 9.0% dry molasses;
about 1% sorbic acid;
about 5.0% brewer's yeast;
about 6.0% sugar;
about 9.5% water;
about 13.0 glycerin;
about 4.0% potato starch;
about 1.0% dehydrated peanut butter;
about 2.0% rice starch; and
about 2.0% guar gum.

In yet another embodiment, the dosage form comprises:
about 3.0-10.0% hemp extract;
about 10.0-20.0% peanut butter;
about 10.0-15.0% rice bran;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 1.0-5.0% rice starch; and
about 1.0-5.0% guar gum.

In yet another embodiment, the dosage form comprises:
about 5.0% hemp extract;
about 15.0% peanut butter;
about 12.5% rice bran;
about 12.75% glucosamine HCl;
about 5.5% sweet potato;
about 8.0% dry molasses;
about 1% sorbic acid;
about 5.0% brewer's yeast;
about 6.0% sugar;
about 9.25% water;
about 13.0 glycerin;
about 2.0% potato starch;
about 1.0% dehydrated peanut butter;
about 2.0% rice starch; and
about 2.0% guar gum.

In yet another embodiment, the dosage form comprises:
about 5.0% hemp extract;
about 15.0% peanut butter;
about 13.0% rice bran;
about 8.5% glucosamine HCl;
about 6.0% sweet potato;
about 9.0% dry molasses;
about 1% sorbic acid;
about 5.0% brewer's yeast;
about 6.0% sugar;
about 9.5% water;
about 13.0 glycerin;
about 4.0% potato starch;
about 1.0% dehydrated peanut butter;
about 2.0% rice starch; and
about 2.0% guar gum.

In yet another embodiment, the dosage form comprises:
about 3.0-10.0% hemp extract;
about 10.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 1.0-5.0% rice starch; and
about 1.0-5.0% guar gum.

In another embodiment, the dosage form further comprises chondroitin sulfate.

In another embodiment, the dosage form comprises 2.0% hemp extract. In another embodiment, the dosage form comprises 3.0% hemp extract. In another embodiment, the dosage form comprises 4.0% hemp extract. In another embodiment, the dosage form comprises 5.0% hemp extract. In another embodiment, the dosage form comprises 6.0% hemp extract. In another embodiment, the dosage form comprises 7.0% hemp extract. In another embodiment, the dosage form comprises 8.0% hemp extract. In another embodiment, the dosage form comprises 9.0% hemp extract. In another embodiment, the dosage form comprises 10.0% hemp extract.

In an embodiment, the hemp extract comprises:
cannabigerolic acid;
cannabigerol;
cannabidiol;
cannabidiolic acid;
Δ9-tetrahydrocannabinol; and
cannabichromene.

In an embodiment, the ratio of cannabigerol to cannabigerolic acid is selected from the group consisting of about 1:100, about 1:50, about 1:10, and about 1:1. In an embodiment, the ratio of cannabigerol to cannabigerolic acid is about 0.1:1 to about 1:0.1. In another embodiment, the ratio of cannabigerol to cannabigerolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabigerol to cannabigerolic acid is about 0.6:1 to about 1:0.6. In yet another embodiment, the ratio of cannabigerol to cannabigerolic acid is about 0.2:1 to about 1:0.2. In yet another embodiment, the ratio of cannabigerol to cannabigerolic acid is about 0.4:1 to about 1:0.4. In still another embodiment, the ratio of cannabigerol to cannabigerolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment. the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 20 mg/mL. In another embodiment, the concentration of Δ9-tetrahydrocannabinol is less than about 10 mg/mL. In another embodiment, the concentration of Δ9-tetrahydrocannabinol is less than about 5 mg/mL. In another embodiment, the concentration of Δ9-tetrahydrocannabinol is less than about 3 mg/mL. In another embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In yet another embodiment, the concentration of Δ9-tetrahydrocannabinol is about 0 mg/mL.

In some embodiments, the concentration of Δ9-tetrahydrocannabinol is less than about 2%. In some embodiments, the concentration of Δ9-tetrahydrocannabinol is less than about 1%. In some embodiments, the concentration of Δ9-tetrahydrocannabinol is less than about 0.5%. In some embodiments, the concentration of Δ9-tetrahydrocannabinol is less than about 0.3%. In some embodiments, the concentration of Δ9-tetrahydrocannabinol is less than about 0.2%. In some embodiments, the concentration of Δ9-tetrahydrocannabinol is less than about 0.1%. In some embodiments, the concentration of Δ9-tetrahydrocannabinol is less than about 0.01%.

In an embodiment, the hemp extract comprises:
about 0.1-20 mg/mL of cannabigerol;
about 0.1-20 mg/mL of cannabigerolic acid;
about 0.01-0.5 mg/mL Δ9-tetrahydrocannabinol; and
about 0.01-0.5 mg/mL cannabichromene.

In another embodiment, the hemp extract comprises:
about 1-10 mg/mL of cannabigerol;
about 1-10 mg/mL of cannabigerolic acid;
less than 0.1 mg/mL Δ9-tetrahydrocannabinol; and
about 0.1-0.4 mg/mL cannabichromene.

In yet another embodiment, the hemp extract comprises:
about 5 mg/mL of cannabigerol;
about 5 mg/mL of cannabigerolic acid;
less than 0.1 mg/mL Δ9-tetrahydrocannabinol; and
about 0.27 mg/mL cannabichromene.

In another embodiment, the hemp extract comprises:
about 1-200 mg/mL of cannabigerol;
about 1-200 mg/mL of cannabigerolic acid;
less than 0.1-5 mg/mL Δ9-tetrahydrocannabinol; and
about 0.1-5 mg/mL cannabichromene.

In another embodiment, the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

In another embodiment, the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

In another embodiment, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In another embodiment, the hemp extract comprises:
about 5% of cannabigerol;
about 5% of cannabigerolic acid;
less than 0.1% Δ9-tetrahydrocannabinol; and
about 0.27% cannabichromene.

In an embodiment, the hemp extract comprises:
about 0.1-20% of cannabigerol;
about 0.1-20% of cannabigerolic acid;
about 0.01-0.5% Δ9-tetrahydrocannabinol; and
about 0.01-0.5% cannabichromene.

In another embodiment, the hemp extract comprises:
about 1-10% of cannabigerol;
about 1-10% of cannabigerolic acid;
less than 0.1% Δ9-tetrahydrocannabinol; and
about 0.1-0.4% cannabichromene.

In yet another embodiment, the hemp extract comprises:
about 5% of cannabigerol;
about 5% of cannabigerolic acid;
less than 0.1% Δ9-tetrahydrocannabinol; and
about 0.27% cannabichromene.

In an embodiment, the hemp extract comprises:
α-pinene;
β-myrcene;
β-pinene;
δ-Limonene;
linalool;
β-caryophyllene;
α-humulene;
nerolidol;
guaiol;
caryophyllene oxide; and
α-bisabolol.

In another embodiment, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol 2;
about 0.02-0.04% guaiol;
about 0.04-0.08% caryophyllene oxide; and
about 0.01-0.04% α-bisabol.

In another embodiment, the hemp extract comprises:
about 0.07-0.30% α-pinene;
about 0.10-0.60% β-myrcene;
about 0.02-0.20% β-pinene;
about 0.03-0.20% δ-limonene;
about 0.01-0.08% linalool;
about 0.03-0.09% β-caryophyllene;
about 0.01-0.06% α-humulene;
about 0.02-0.09% nerolidol; and
about 0.01-0.06% guaiol.

In another embodiment, the hemp extract comprises:
about 0.01-0.50% α-pinene;
about 0.01-0.90% β-myrcene;
about 0.01-0.50% β-pinene;
about 0.01-0.50% δ-limonene;
about 0.01-0.50% linalool;
about 0.01-0.50% β-caryophyllene;
about 0.01-0.50% α-humulene;
about 0.01-0.50% nerolidol;
about 0.01-0.50% guaiol;
about 0.01-0.50% caryophyllene oxide; and
about 0.01-0.50% α-bisabolol.

In another embodiment, the hemp extract further comprises:
camphene;
β-ocimene;
eucalyptol;
isopulegol; and/or
nerolidol.

In another embodiment, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

In another embodiment, the hemp extract comprises:
about 0.01-0.04% camphene;
about 0.01-0.05% β-ocimene;
about 0.01-0.07% eucalyptol;
about 0.01-0.04% isopulegol; and/or
about 0.01-0.05% nerolidol.

In another embodiment, the hemp extract comprises:
about 0.01-0.50% camphene;
about 0.01-0.50% β-ocimene;
about 0.01-0.50% eucalyptol;
about 0.01-0.50% isopulegol; and/or
about 0.01-0.50% nerolidol.

In an embodiment, the composition is formulated as an oil. In another embodiment, the carrier is selected from the group consisting of linseed oil, olive oil, fish oil, salmon oil, coconut oil, catnip oil, sesame oil, MCT oil, and grapeseed oil. In yet another embodiment, the carrier comprises grapeseed oil. In yet another embodiment, the carrier comprises sesame oil.

In an embodiment, the flavoring agent is selected from the group consisting of catnip oil, chicken liver powder, poultry extract, maltodextrin, butter, and bacon. In another embodiment, the flavoring agent is chicken liver powder.

In an embodiment, the composition is formulated as a chew for oral administration. In another embodiment, the chew is produced using cold extrusion In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In an embodiment, the 4 g chew comprises:
about 7 mg of cannabigerol;
about 6 mg of cannabigerolic acid;
about 0.32 mg Δ9-tetrahydrocannabinol; and
about 0.36 mg cannabichromene.

In some embodiments, the chew comprises:
about 70 mg of cannabigerol;
about 60 mg of cannabigerolic acid;
about 3.2 mg Δ9-tetrahydrocannabinol; and
about 3.6 mg cannabichromene.

### Methods of Treatment

In an aspect, provided herein is a method for treating or reducing pain in a veterinary subject in need thereof, comprising administering to the subject a therapeutically effective amount of any of the compositions or dosage forms described above.

In an embodiment, the pain is associated with arthritis, post-operative pain, acute pain, dental pain, pain associated with gingivitis, joint pain, or multi-joint pain.

In an embodiment, the veterinary subject has cancer. In an embodiment, the cancer is a solid tumor, such as lung cancer, prostate cancer, colorectal cancer, thyroid cancer, renal cancer, adrenal cancer, liver cancer, pancreatic cancer, mammary cancer and central and peripheral nervous system cancer. In another embodiment, the cancer is a hematopoietic tumor, such as lymphomas and leukemias.

In an embodiment, the veterinary subject is undergoing chemotherapy. In an embodiment, the chemotherapy is L-asparaginase, cyclophosphamide, doxorubicin, vincristine. and prednisolone (L-CHOP) or cyclophosphamide, doxorubicin, vincristine, and prednisolone (CHOP).

In an embodiment, the method results in a reduced tumor burden. In another embodiment, the method results in apoptosis of tumor cells. In another embodiment, the method results in a decrease in the proliferation of tumor cells.

In an embodiment, the pharmaceutical composition or dosage form is administered at a dosage of about 0.1-15.0 mg/kg. In another embodiment, the pharmaceutical composition or dosage form is administered at a dosage of about 0.1-10.0 mg/kg. In yet another embodiment, the pharmaceutical composition or dosage form is administered at a dosage of about 0.1 mg/kg. In still another embodiment, the pharmaceutical composition or dosage form is administered at a dosage of about 0.2 mg/kg. In yet another embodiment, the pharmaceutical composition or dosage form is administered at a dosage of about 0.3 mg/kg. In an embodiment, the pharmaceutical composition or dosage form is administered at a dosage of about 0.4 mg/kg. In another embodiment, the pharmaceutical composition or dosage form is administered at a dosage of about 0.5 mg/kg. In yet another embodiment, the pharmaceutical composition or dosage form is administered at a dosage of about 0.6 mg/kg. In still another embodiment, the pharmaceutical composition or dosage form is administered at a dosage of about 0.7 mg/kg. In yet another embodiment, the pharmaceutical composition or dosage form is administered at a dosage of about 0.8 mg/kg. In an embodiment, the pharmaceutical composition or dosage form is administered at a dosage of about 0.9 mg/kg. In another embodiment, the pharmaceutical composition or dosage form is administered at a dosage of about 1 mg/kg. In yet another embodiment, the pharmaceutical composition or dosage form is administered at a dosage of about 1.5 mg/kg. In still another embodiment, the pharmaceutical composition or dosage form is administered at a dosage of about 2 mg/kg. In an embodiment, the pharmaceutical composition or dosage form is administered at a dosage of about 3 mg/kg. In another embodiment, the pharmaceutical composition or dosage form is administered at a dosage of about 4 mg/kg. In yet another embodiment, the pharmaceutical composition or dosage form is administered at a dosage of about 5 mg/kg. In still another embodiment, the pharmaceutical composition or dosage form is administered at a dosage of about 6 mg/kg. In an embodiment, the pharmaceutical composition or dosage form is administered at a dosage of about 7 mg/kg. In another embodiment, the pharmaceutical composition or dosage form is administered at a dosage of about 8 mg/kg. In yet another embodiment, the pharmaceutical composition or dosage form is administered at a dosage of about 9 mg/kg. In still another embodiment, the pharmaceutical composition or dosage form is administered at a dosage of about 10 mg/kg. In an embodiment, the pharmaceutical composition or dosage form is administered at a dosage of about 11 mg/kg. In another embodiment, the pharmaceutical composition or dosage form is administered at a dosage of about 12 mg/kg. In yet another embodiment, the pharmaceutical composition or dosage form is administered at a dosage of about 13 mg/kg. In still another embodiment, the pharmaceutical composition or dosage form is administered at a dosage of about 14 mg/kg. In an embodiment, the pharmaceutical composition or dosage form is administered at a dosage of about 15 mg/kg.

In another embodiment, the pharmaceutical composition or dosage form is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the pharmaceutical composition or dosage form is administered at a dosage of about 1 mg/kg for one week, and then subsequently administered at a dosage of about 0.1-0.5 mg/kg. In another embodiment, the pharmaceutical composition or dosage form is administered at a dosage of about 4 mg/kg for one week, and then subsequently administered at a dosage of about 2 mg/kg.

In an embodiment, the pharmaceutical composition or dosage form is administered at a dosage of about 2 mg/kg every 12 hours for two weeks, then subsequently administered at a dosage of about 1 mg/kg every 12 hours for two weeks, and then subsequently administered at a dosage of about 2 mg/kg every 12 hours for four weeks.

In an embodiment, the pharmaceutical composition or dosage form is administered at a dosage of about 1.0 mg/kg once daily. In an embodiment, the pharmaceutical composition or dosage form is administered at a dosage of about 1.0 mg/kg twice daily. In an embodiment, the pharmaceutical composition or dosage form is administered at a dosage of about 1.0 mg/kg three times daily. In an embodiment, the pharmaceutical composition or dosage form is administered at a dosage of about 1.0 mg/kg four times daily.

In an embodiment, the pharmaceutical composition or dosage form is administered at a dosage of about 2.0 mg/kg once daily. In an embodiment, the pharmaceutical composition or dosage form is administered at a dosage of about 2.0 mg/kg twice daily. In an embodiment, the pharmaceutical composition or dosage form is administered at a dosage of about 2.0 mg/kg three times daily. In an embodiment, the pharmaceutical composition or dosage form is administered at a dosage of about 2.0 mg/kg four times daily.

In an embodiment, the pharmaceutical composition or dosage form is administered at a dosage of about 3.0 mg/kg once daily. In an embodiment, the pharmaceutical composition or dosage form is administered at a dosage of about 3.0 mg/kg twice daily. In an embodiment, the pharmaceutical composition or dosage form is administered at a dosage of about 3.0 mg/kg three times daily. In an embodiment, the pharmaceutical composition or dosage form is administered at a dosage of about 3.0 mg/kg four times daily.

In an embodiment, the pharmaceutical composition or dosage form is administered at a dosage of about 4.0 mg/kg once daily. In an embodiment, the pharmaceutical composition or dosage form is administered at a dosage of about 4.0 mg/kg twice daily. In an embodiment, the pharmaceutical composition or dosage form is administered at a dosage of about 4.0 mg/kg three times daily. In an embodiment, the pharmaceutical composition or dosage form is administered at a dosage of about 4.0 mg/kg four times daily. In an embodiment, the pharmaceutical composition or dosage form is administered at a dosage of about 5.0 mg/kg once daily. In an embodiment, the pharmaceutical composition or dosage form is administered at a dosage of about 5.0 mg/kg twice daily. In an embodiment, the pharmaceutical composition or dosage form is administered at a dosage of about 5.0 mg/kg three times daily. In an embodiment, the pharmaceutical composition or dosage form is administered at a dosage of about 5.0 mg/kg four times daily.

In an embodiment, the pharmaceutical composition or dosage form is administered at a dosage of about 6.0 mg/kg once daily. In an embodiment, the pharmaceutical composition or dosage form is administered at a dosage of about 6.0 mg/kg twice daily. In an embodiment, the pharmaceutical composition or dosage form is administered at a dosage of about 6.0 mg/kg three times daily. In an embodiment, the pharmaceutical composition or dosage form is administered at a dosage of about 6.0 mg/kg four times daily.

In an embodiment, the pharmaceutical composition or dosage form is administered at a dosage of about 7.0 mg/kg once daily. In an embodiment, the pharmaceutical composition or dosage form is administered at a dosage of about 7.0 mg/kg twice daily. In an embodiment, the pharmaceutical composition or dosage form is administered at a dosage of about 7.0 mg/kg three times daily. In an embodiment, the pharmaceutical composition or dosage form is administered at a dosage of about 7.0 mg/kg four times daily.

In an embodiment, the pharmaceutical composition or dosage form is administered at a dosage of about 8.0 mg/kg once daily. In an embodiment, the pharmaceutical composition or dosage form is administered at a dosage of about 8.0 mg/kg twice daily. In an embodiment, the pharmaceutical composition or dosage form is administered at a dosage of about 8.0 mg/kg three times daily. In an embodiment, the pharmaceutical composition or dosage form is administered at a dosage of about 8.0 mg/kg four times daily.

In an embodiment, the pharmaceutical composition or dosage form is administered at a dosage of about 9.0 mg/kg once daily. In an embodiment, the pharmaceutical composition or dosage form is administered at a dosage of about 9.0 mg/kg twice daily. In an embodiment, the pharmaceutical composition or dosage form is administered at a dosage of about 9.0 mg/kg three times daily. In an embodiment, the pharmaceutical composition or dosage form is administered at a dosage of about 9.0 mg/kg four times daily.

In an embodiment, the pharmaceutical composition or dosage form is administered at a dosage of about 10.0 mg/kg once daily. In an embodiment, the pharmaceutical composition or dosage form is administered at a dosage of about 10.0 mg/kg twice daily. In an embodiment, the pharmaceutical composition or dosage form is administered at a dosage of about 10.0 mg/kg three times daily. In an embodiment, the pharmaceutical composition or dosage form is administered at a dosage of about 10.0 mg/kg four times daily.

In an embodiment, the method results in a therapeutically effective median maximal serum concentration of cannabigerol. In another embodiment, the median maximal serum concentration of cannabigerol is about 20-200 ng/mL. In another embodiment, the median maximal serum concentration of cannabigerol is about 30-80 ng/mL. In another embodiment, the median maximal serum concentration of cannabigerol is about 30-60 ng/mL. In another embodiment, the median maximal serum concentration of cannabigerol is about 40-50 ng/mL. In another embodiment, the median maximal serum concentration of cannabigerol is about 50-200 ng/mL. In another embodiment, the median maximal serum concentration of cannabigerol is about 75-200 ng/mL. In another embodiment, the median maximal serum concentration of cannabigerol is about 100-200 ng/mL.

In an embodiment, the method results in a therapeutically effective median maximal serum concentration of cannabigerolic acid. In another embodiment, the median maximal serum concentration of cannabigerolic acid is about 1000-4000 ng/mL. In another embodiment, the median maximal serum concentration of cannabigerolic acid is about 1000-3000 ng/mL. In another embodiment, the median maximal serum concentration of cannabigerolic acid is about 1500-3000 ng/mL. In another embodiment, the median maximal serum concentration of cannabigerolic acid is about 1200-2200 ng/mL. In another embodiment, the median maximal serum concentration of cannabigerolic acid is about 2000-3000 ng/mL. In another embodiment, the median maximal serum concentration of cannabigerolic acid is about 3000-4000 ng/mL.

In an embodiment, the veterinary subject is canine, feline, bovine, porcine, or equine. In another embodiment, the veterinary subject is canine. In yet another embodiment, the veterinary subject is feline.

In an aspect, provided herein is a method for treating or reducing pain associated with arthritis, post-operative pain, acute pain, dental pain, pain associated with gingivitis, joint pain, or multi-joint pain in a veterinary subject in need thereof, comprising administering to the subject a therapeutically effective amount of hemp extract.

In an embodiment, the hemp extract is administered at a dosage of about 0.1-15.0 mg/kg. In another embodiment, the hemp extract is administered at a dosage of about 0.1-10.0 mg/kg. In yet another embodiment, the hemp extract is administered at a dosage of about 0.1 mg/kg. In still another embodiment, the hemp extract is administered at a dosage of about 0.2 mg/kg. In yet another embodiment, the hemp extract is administered at a dosage of about 0.3 mg/kg. In an embodiment, the hemp extract is administered at a dosage of about 0.4 mg/kg. In another embodiment, the hemp extract is administered at a dosage of about 0.5 mg/kg. In yet another embodiment, the hemp extract is administered at a dosage of about 0.6 mg/kg. In still another embodiment, the hemp extract is administered at a dosage of about 0.7 mg/kg. In yet another embodiment, the hemp extract is administered at a dosage of about 0.8 mg/kg. In an embodiment, the hemp extract is administered at a dosage of about 0.9 mg/kg. In another embodiment, the hemp extract is administered at a dosage of about 1 mg/kg. In yet another embodiment, the hemp extract is administered at a dosage of about 1.5 mg/kg. In still another embodiment, the hemp extract is administered at a dosage of about 2 mg/kg. In an embodiment, the hemp extract is administered at a dosage of about 3 mg/kg. In another embodiment, the hemp extract is administered at a dosage of about 4 mg/kg. In yet another embodiment, the hemp extract is administered at a dosage of about 5 mg/kg. In still another embodiment, the hemp extract is administered at a dosage of about 6 mg/kg. In an embodiment, the hemp extract is administered at a dosage of about 7 mg/kg. In another embodiment, the hemp extract is administered at a dosage of about 8 mg/kg. In yet another embodiment, the hemp extract is administered at a dosage of about 9 mg/kg. In still another embodiment, the hemp extract is administered at a dosage of about 10 mg/kg. In an embodiment, the hemp extract is administered at a dosage of about 11 mg/kg. In another embodiment, the hemp extract is administered at a dosage of about 12 mg/kg. In yet another embodiment, the hemp extract is administered at a dosage of about 13 mg/kg. In still another embodiment, the hemp extract is administered at a dosage of about 14 mg/kg. In an embodiment, the hemp extract is administered at a dosage of about 15 mg/kg.

In another embodiment, the hemp extract is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the hemp extract is administered at a dosage of about 1 mg/kg for one week, and then subsequently administered at a dosage of about 0.1-0.5 mg/kg. In another embodiment, the hemp extract is administered at a dosage of about 4 mg/kg for one week, and then subsequently administered at a dosage of about 2 mg/kg.

In an embodiment, the veterinary subject is administered gabapentin in combination with a dosage of hemp extract provided herein. In other embodiments, the veterinary subject is administered a dosage of about of about 1, 10, 15, 20, 25, 30, 35, 40, 45 or 50 mg/kg of gabapentin with a dosage of hemp extract provided herein. In another embodiment, the veterinary subject is administered a dosage of about 10 mg/kg of gabapentin with a dosage of hemp extract provided herein.

In another embodiment, the veterinary subject is administered a dosage of from 1 mg/kg to 50 mg/kg of gabapentin with a dosage of hemp extract provided herein. In another embodiment, the veterinary subject is administered a dosage from 10 mg/kg to 40 mg/kg of gabapentin with a dosage of hemp extract provided herein. In another embodiment, the veterinary subject is administered a dosage from 1 mg/kg to 20 mg/kg of gabapentin with a dosage of hemp extract provided herein. In another embodiment, the veterinary subject is administered a dosage from 5 mg/kg to 15 mg/kg of gabapentin with a dosage of hemp extract provided herein. In another embodiment, the veterinary subject is administered a dosage from 12 mg/kg to 14 mg/kg of gabapentin with a dosage of hemp extract provided herein.

In some embodiments, the veterinary subject is administered gabapentin about every 1, 2, 3, 4, 5, 6, 7, 8, 9, 10, 11 or 12 hours with a dosage of hemp extract provided herein. In one embodiment, the veterinary subject is administered gabapentin about every 8 hours with a dosage of hemp extract provided herein. In one embodiment, the veterinary subject is administered gabapentin every 8 to 12 hours with a dosage of hemp extract provided herein. The hemp extract can be administered with the gabapentin or at a different time and/or on a different schedule.

In another embodiment, the veterinary subject is administered gabapentin 1, 2, 3, 4, 5 or 6 times daily with a dosage of hemp extract provided herein. The hemp extract can be administered with the gabapentin or at a different time and/or on a different schedule.

In an embodiment, the veterinary subject is administered about 10 mg/kg gabapentin about every 8 hours with a dosage of hemp extract provided herein. The hemp extract can be administered with the gabapentin or at a different time and/or on a different schedule. In another embodiment, the veterinary subject is administered 10 mg/kg gabapentin and 8 mg/kg hemp extract every 8 hours. The hemp extract can be administered with the gabapentin or at a different time.

In an embodiment, the veterinary subject is canine, feline, bovine, porcine, or equine. In another embodiment, the veterinary subject is canine. In yet another embodiment, the veterinary subject is feline.

The pharmaceutical compositions and dosage forms of the present disclosure may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local. In an embodiment, administration is topical. In another embodiment, topical administration is used to treat local pain. In another embodiment, the local pain is joint pain. In an embodiment, the veterinary subject is an animal >100 kg (e.g., a horse, cow, or pig).

The therapeutic compositions of the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the weight of a subject to be administered, target disease, conditions, route of administration, and the like. Various delivery systems are known and can be used to administer the pharmaceutical composition of the invention, e.g., encapsulation in liposomes, microparticles, microcapsules, receptor mediated endocytosis (see, e.g., Wu et al. (1987) J. Biol. Chem. 262:4429-4432). Methods of introduction include, but are not limited to, intradermal, intramuscular, intraperitoneal, intravenous, transdermal, buccal, sublingual, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the pharmaceutical composition or dosage form in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as tale or magnesium stearate and, optionally, stabilizers. In soft capsules, the active components may be dissolved or suspended in suitable liquids, such as fatty oils, liquid paraffin, or liquid polyethylene glycols.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

Advantageously, the pharmaceutical compositions for oral or parenteral use described above are prepared into dosage forms in a unit dose suited to fit a dose of the active ingredients. Such dosage forms in a unit dose include, for example. tablets, pills, capsules, injections (ampoules), suppositories, chews, pet food, etc. In certain embodiments, for the dosages provided above, they are administered in one serving of pet food, e.g. 1 mg/kg of hemp extract provided in one serving of pet food.

In accordance with the methods disclosed herein, pharmaceutical formulations can be administered to the patient using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer a pharmaceutical formulation.

In an embodiment for non-human animal administration, the term “pharmaceutical” as used herein may be replaced by “veterinary.”

### EXAMPLES

### Example 1

### Canine Pharmacokinetic and Safety Study

A study was conducted to evaluate the pharmacokinetics of CBGA and CBG using a 24-hour pharmacokinetic analysis and a determination of steady state levels after 2 weeks of treatment in both the fed and fasted state. Second, the study evaluated the safety of these compounds in the dog over the two-week trial using physical examination (e.g., heart rate), complete blood counts and serum chemistry evaluations.

### Animals and Protocol

Six intact male beagles all 2 years of age weighing between 18.6 and 22.2 kilograms body weight were housed at the Baker Institute for Animal Health and all procedures were approved by the Cornell University Institutional Animal Care and Use Committee (Protocol 2019-0001). All dogs underwent the fasted treatment trial for a two-week period and were then allowed 2 weeks of washout before beginning the fed stage of the trial which included ¼ of a 13 ounce can of a wet food (PURINA® Pro Plan Savory Chicken and Rice Formula, Nestle Purina, St. Louis, MO) at the time of dosing. Twenty-four hour pharmacokinetic analysis of serum cannabinoid levels was performed on day 1 after a single dose in the fed or fasted state. Then, twice daily dosing of treatment oil at both 7 am and 7 pm was performed on the following days for two weeks, and serum cannabinoids were measured. Prior to the study all dogs had blood drawn for a complete blood count and serum chemistry and again at the end of each phase of the 2 week study. Complete blood counts (white blood cells [WBC], hematocrit, hemoglobin, red blood cells [RBC], neutrophils, lymphocytes, platelets, monocytes, eosinophils, basophils) and serum biochemistry analyses (sodium, potassium, chloride, magnesium, calcium, phosphorus, albumin, total protein, globulin, urea nitrogen, creatinine, alkaline phosphatase [ALP], alanine aminotransferase [ALT], aspartate amino transferase [AST], cholesterol, total bilirubin, glucose and gamma glutamyl transferase [GGT]) were performed at the Cornell University Diagnostic Laboratory. For the fasted and fed portions of the 24 hour pharmacokinetic analysis study the dogs were administered 2 mg/kg body weight of an equal mix of 30 mg/mL CBG and 30 mg/mL CBGA from a hemp extract in a sesame oil base which was tested again at the end of the study showing virtually the same analytical profile. Two milliliters of blood was drawn via jugular venipuncture for all 6 dogs prior to the initial dose (0 hours) and then again at 0.5, 1, 2, 4, 8, 12 and 24 hours. Prior to enrollment all dogs involved in the study were deemed healthy based on physical examination. Behavioral observations were performed 2 hours after morning dosing for each phase of the trial. Behavioral observations were performed on day 1, day 3, day 7, and day 14. Physical and behavioral examinations were performed to assess for adverse events including diarrhea, vomiting, lethargy, somnolence, ataxia, or abnormal behavior within the colony. Due to the potential for CBG/CBGA to affect heart rate, dogs were assessed manually for resting heart rates for 30 seconds and extrapolated to relative beats per minute after initial dosing at time points 0, 1, 2, 4, 12, 24 and 48 hours and again at day 7 and day 14 after morning dosing for both the fed and fasted arms of the study. Serum was also obtained on day 7 and day 14 six hours after the morning dose for further serum cannabinoid measurement to establish relative steady state serum concentrations.

### Serum Cannabinoids Analysis

Analysis was performed using an exploratory (fit-for-purpose) method for fast measurement of thirteen cannabinoids and their metabolites at the Toxicology Research Laboratory, University of Illinois at Chicago. The reference standards for CBD and CBDA were obtained from Restek Corporation (Bellefonte, PA); all other reference and internal standards including CBG and CBGA were obtained from Cerilliant Corporation (Round Rock, TX). The concentrations of cannabinoids (CBD, CBDA, 7-COOH Cannabidiol (7-COOH-CBD), THC, THCA, cannabinol (CBN), cannabichromene (CBC), CBG, and CBGA) and their metabolites (7-OH-CBD, 7-COOH-CBD, COOH-THC, and COOH-THC-Glu) in dog sera were determined using high performance liquid chromatography with tandem mass spectrometry (LC-MS/MS) (Nexera X2 and LCMS 8050, Shimadzu Corp., Kyoto, Japan).

Dog serum (40 μL) was mixed with 20 μL of internal standards [100 ng/mL of CBD-d3, THC-d3, THCA-d3, 7-COOH-CBD-d3, COOH-THC-d9, and COOH-THC-Glu-d3 in water:methanol (50:50)] in a 96 well plate. Proteins were precipitated and compounds were extracted by adding 100 μL of ice-cold acetonitrile to the samples, then vortexing for 1-2 minutes and centrifuging at 4,000 rpm for 10 minutes at 4° C. Supernatants (70 μL) were mixed with 70 μL of water in a different 96 well plate and centrifuged again. 10 μL of the processed samples were injected into a WATERS™ ATLANTIS® T3 HPLC column (3 pm 2.1×50 mm) with a guard cartridge (WATERS™ VanGuard ATLANTIS® T3) coupled to LC-MS/MS (Waters Corporation, Milford, Massachusetts, United States). The column was equilibrated with mobile phase A (0.1% formic acid in water) and mobile phase B (acetonitrile) at 50% B. The compounds were eluted by a linear gradient from 50% B to 95% B over 6 minutes, and then held at 95% B for 1 minute. Subsequently, the column was re-equilibrated at initial composition for 1 minute at a flow rate of 0.3 mL/min. The autosampler and column temperature were set a 4 and 30° C., respectively. The compounds were detected in electrospray ionization positive and/or negative mode. Interface voltage and temperature were 4 kV and 300° C., respectively. Desolvation line and heat block temperatures were 250 and 400° C., respectively. Nebulizing, heating, and drying gas flow were 2.7, 5, and 5 L/min, respectively.

Concentrations of cannabinoids were calculated by LabSolutions software (Shimadzu Corp., Kyoto, Japan) using a quadratic calibration curve with 1/c² weighing based on relative response (peak area of cannabinoids/peak area of internal standards).

### Pharmacokinetics and Statistical Analysis

The 24-hour non-compartmental pharmacokinetic analysis of CBG and CBGA was performed utilizing a commercial software system. (PK solutions 2.0, Summit PK, Montrose, CO). Semi-log plots were utilized to determine linearity of the elimination profiles. The results generated were time to maximal concentrations (Tmax), maximum serum concentration (Cmax), elimination half-life (T ½), area under the curve to the last time-point (AUC₀₋₂₄), and mean residence time (MRT). The program predicts steady state average, minimal and maximal and average concentrations with chronic administration based on the assumption that steady state is achieved after five half-lives of administration (Css Ave).

Statistical analysis of the pharmacokinetic results was performed on fed versus fasted 24-hour pharmacokinetics using a Student's T Test to determine whether there were significant differences. Similarly, complete blood count data and selected serum chemistry data (hepatic and kidney parameters) from the dogs in each phase were compared at baseline and 2 weeks for both the fed and fasted portions of study using a Student's T Test. A one way analysis of variation was performed on heart rate assessments over time for each phase of the study (fed and fasted). A two way analysis of variance was performed on the week 1 and week 2 fed and fasted data to assess any differences between the two groups over time. For all statistical testing the p value was set at ≤0.05 for significance. All statistical testing was performed with GraphPad Prism 6.0 (Graphpad, LaJolla, CA), and figures were generated by the same software. Any result that was below the quantifiable limit for the respective cannabinoid was considered 0 for all graphing and representation of data (CBG lower limit 0.5 ng/mL; CBGA lower limit 1 ng/mL).

### Results

### 24 Hour, 1 Week, and 2 Week Pharmacokinetics

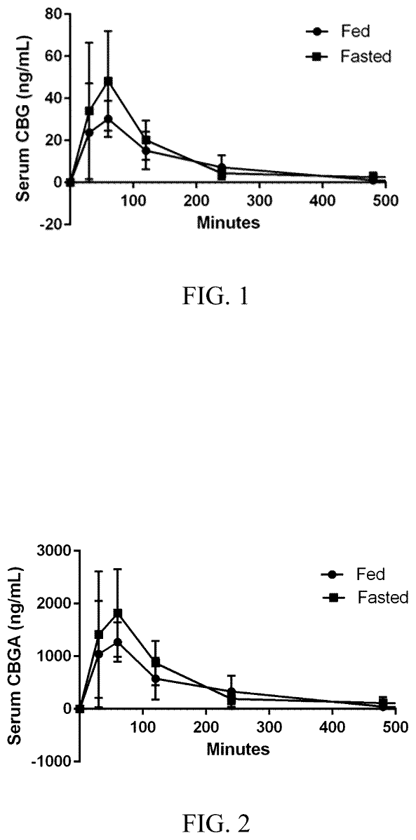

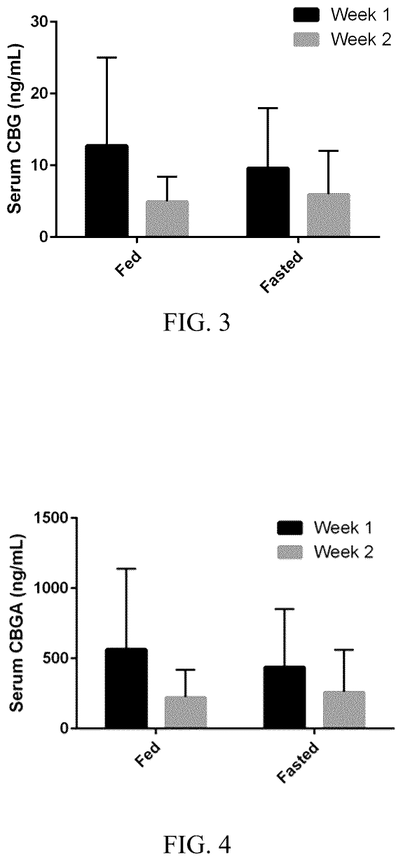

Twenty four hour PK analyses resulted in calculable Cmax (ng/mL), Tmax (h), elimination T½ life (h), AUC 0-24 (ng-h/mL), MRT (h), and predicted steady state average means for CBG and CBGA. No other cannabinoid could be found at the detectable ranges. All data, means, and standard deviations for CBG as well as CBGA in the fed and fasted state are found in Table 1 and 2, respectively. There were no statistically significant differences noted across all pharmacokinetic parameters when comparing the fed and fasted results (Table 1). The CBGA serum concentrations were far higher than found for CBG globally across all dogs and although not statistically significant, both CBG and CBGA concentrations are higher in the fasted state (FIG. 1 and FIG. 2). No significant differences in CBG serum concentrations were observed at week one in the fed (12.7±12.3 ng/mL) or fasted (5.0±3.5 ng/mL) state. Similarly, CBG serum concentrations were not significantly different at week two between the fed (9.6±8.4 ng/mL) and fasted (6.1±6.0 ng/mL) state, respectively (FIG. 3). No significant differences for CBG between week, or fed and fasted state could be found (time p=0.65, fed/fasted p=0.10; time*fed/fasted p=0.46). One and two week 6 hour post morning dose of oil showed that CBGA serum concentrations were 564±574 ng/mL and 223±197 ng/mL in the fed state, respectively and 437±414 ng/mL and 260±300 ng/mL in the fasted state, respectively (FIG. 4). No significant differences for CBGA between week, or fed and fasted state could be found (time p=0.11, fed/fasted p=0.69; time*fed/fasted p=0.48). All of these results are of a similar order to predicted steady state concentrations observed from the pharmacokinetic analysis.

TABLE 1
Mean and standard deviations (n = 6) on serum 24-hour pharmacokinetic
values of CBG fed and fasted states. P values at bottom represent comparisons
between fed and fasted states.

| | Cmax | Tmax | T½ | AUC | MRT | Css Ave |
|---|---|---|---|---|---|---|
| | (ng/mL) | (h) | el (h) | (0-24 ng-h/mL) | (hr) | (ng/mL) |
| CBG Fed | | | | | | |
| Beagle 1 | 20.5 | 1 | 1.99 | 61.9 | 3.2 | 6.3 |
| Beagle 2 | 42.6 | 0.5 | 2.44 | 117.2 | 3.4 | 8.4 |
| Beagle 3 | 38.3 | 0.5 | 2.17 | 123.9 | 3.2 | 9.8 |
| Beagle 4 | 52.9 | 0.5 | 1.36 | 66.3 | 1.7 | 5.8 |
| Beagle 5 | 33.6 | 1 | 1.45 | 49.4 | 2.3 | 5.3 |
| Beagle 6 | 46.5 | 1 | 1.38 | 81.5 | 2.4 | 9.3 |
| Mean ± SD | 39.1 ± 11.3 | 0.75 ± 0.27 | 1.80 ± 0.46 | 83.4 ± 30.6 | 2.7 ± 0.7 | 7.5 ± 1.9 |
| CBG Fasted | | | | | | |
| Beagle 1 | 85.4 | 0.5 | 2.9 | 166.4 | 4 | 14.7 |
| Beagle 2 | 26 | 2 | 2.22 | 83.2 | 3.4 | 8.2 |
| Beagle 3 | 55.4 | 0.5 | 2.22 | 106.1 | 3.1 | 9.6 |
| Beagle 4 | 43.6 | 1 | 2.18 | 100.5 | 3.1 | 9.4 |
| Beagle 5 | 43.9 | 1 | 1.39 | 63.7 | 1.8 | 5.4 |
| Beagle 6 | 73 | 1 | 1.54 | 73 | 2.1 | 11.6 |
| Mean ± SD | 54.7 ± 21.5 | 1 ± 0.5 | 2.1 ± 0.5 | 98.8 ± 36.8 | 2.9 ± 0.8 | 9.8 ± 3.1 |
| P value | 0.25 | 0.41 | 0.21 | 0.48 | 0.50 | 0.15 |

Cmax, Maximum serum concentration
Tmax, Time to a mximal concentration
T½, Half-life of elimination

TABLE 2
Mean and standard deviation concentrations (n = 6) on serum 24-pharmacokinetics
values of CBGA fed and fasted states. P values at bottom represent
comparisons between fed and fasted states

| | Cmax | Tmax | | AUC (0-24 | MRT | Css Ave |
|---|---|---|---|---|---|---|
| | (ng/ml) | (h) | T½ el (h) | ng-h/mL) | (hr) | (ng/ml) |
| CBG Fed | | | | | | |
| Beagle 1 | 737 | 1 | 1.99 | 2275 | 3.3 | 228 |
| Beagle 2 | 1866 | 0.5 | 2.27 | 3722 | 2.8 | 361 |
| Beagle 3 | 1816 | 0.5 | 1.01 | 5469 | 2.2 | 759 |
| Beagle 4 | 2191 | 0.5 | 1.69 | 2890 | 1.9 | 243 |
| Beagle 5 | 1336 | 1 | 1.59 | 1890 | 2.2 | 180 |
| Beagle 6 | 1890 | 1 | 1.32 | 3224 | 2.3 | 353 |
| Mean ± SD | 1639 ± 521 | 0.75 ± 0.27 | 1.64 ± 0.45 | 3,245 ± 1271 | 2.5 ± 0.5 | 353.8 ± 211.0 |
| CBG Fasted | | | | | | |
| Beagle 1 | 2998.6 | 0.5 | 3.43 | 5580 | 5.2 | 528 |
| Beagle 2 | 1082.3 | 2 | 2.27 | 3648 | 3.5 | 351 |
| Beagle 3 | 2482.9 | 0.5 | 2.11 | 4752 | 2.9 | 420 |
| Beagle 4 | 1891.8 | 1 | 2.18 | 4589 | 3.1 | 429 |
| Beagle 5 | 1638.9 | 1 | 1.75 | 2460 | 1.9 | 184 |
| Beagle 6 | 3352.1 | 1 | 1.36 | 5588 | 1.9 | 520 |
| Mean ± SD | 2241.1 ± 859.5 | 1 ± 0.5 | 2.2 ± 0.7 | 4436 ± 1207 | 3.1 ± 1.2 | 405 ± 127 |
| P Value | 0.25 | 0.41 | 0.08 | 0.12 | 0.14 | 0.60 |

Cmax, Maximum serum concentration
Tmax, Time to a maximal concentration
T½, Half-life of elimination
AUC (0-24), Area under the serum concentration curve to 24 hrs
MRT, Mean Residence Time
Css Ave, Predicted average steady state serum concentration
No significant differences found between Fed and Fasted States.

### Serum Chemistry and Complete Blood Counts

Mean and standard deviation serum chemistry and complete blood counts at baseline and week 2 are shown in Tables 3 and 4 respectively. Serum chemistry changes associated with treatment with the cannabinoid-and-oil-based-product suggests that there were very mild increases in the fed and fasted state for serum creatinine (p<0.01 fasted; p<0.01 fed) with no values falling outside of the reference range. Serum albumin (p=0.03), cholesterol (p=0.03) and AST (p=0.01) levels also shows a significant increase during the fed phase at week 2, while serum ALP (fasted p=0.02; fed p=0.05) shows a decrease during the fed and fasted phases at week 2. None of these increases or decreases fell outside of the reference ranges for each parameter. No other significances were noted among remaining kidney and hepatic parameters. Serum electrolytes showed no significant differences across time with treatment (data not shown).

Complete blood counts data were noted for white blood cell counts with higher counts at week 2 during both the fed and fasted states (fasted p=0.02; fed p=0.01). Similarly, when examining specific cell types, neutrophils were found to be increased at week 2 after each phase of treatment (fasted p=0.02; fed p=0.01). Platelets and monocytes were significantly increased at week 2 during the fasted phase of the trial (p<0.01). Hematocrit concentration was found to be significantly increased in the fed state 2 weeks after treatment (p=0.01). No parameters were above or below the reference range intervals.

Physical examinations performed throughout found no observable abnormalities regarding activity, neurological deficits, or behavior at any stage of the study. Vomiting was noted in 1 dog on day two after morning dosing of the fasting phase with no other changes in appetite, vomiting or diarrhea. The heart rate observed during the two-week period across both phases of the trial are found in Table 5. No significant differences were noted across the fed and fasted phases of the trial.

### CONCLUSIONS AND DISCUSSION

This is the first pharmacokinetic serum assessment of CBG and CBGA using an infused sesame oil that contained a mixture of 30 mg/ml CBG and 30 mg/ml CBGA given orally to dogs. Cultivars that specifically synthesize primarily CBG or its acid derivative are likely to be developed for nutraceutical sales. It is important to evaluate the CBG and CBGA in companion animals, such as the dog, to better understand the pharmacokinetics and utility of these products, particularly in light of NSAID intolerance and the need for safe alternatives for pain control.

Our 24-hour pharmacokinetic examination of CBG and CBGA demonstrates a marked increase in CBGA concentrations when compared to CBG in dog serum. CBGA levels in the serum show approximately 40 times higher Cmax when compared to CBG in both the fed and fasted state, with similar Tmax and T ½ lives for both compounds. In the limited human literature, there is little focus on the acidic forms and oral consumption makes this study novel in a higher mammalian species examining the pharmacokinetics of CBG and CBGA showing definitive superiority in absorption and retention of CBGA.

The pharmacokinetics of CBG and CBGA over a 2-week period shows absorption and retention after oral dosing in both the fasted or fed state. However, the CBGA is absorbed at a much higher concentration and maintains a far higher serum concentration when compared to CBG. Comparatively, this makes CBGA a better pharmacologic molecule since its absorption and retention appear to be nearly 10-fold higher than what can be achieved with CBG. Although there were no significant differences between the weeks or fed/fasted states of CBG nor CBGA in our small study the p value over time for CBGA was 0.11 which points towards potential differences over time, which may or may not be clinically relevant. Interestingly, week two concentrations are lower than week one regardless of the fed or fasted state suggesting that there may be alterations in hepatic metabolism that occur at the initiation of dosing which may lead to the disposal of CBG in urine or bile that warrant further investigation of cytochrome p450 enzymatic induction or inhibition.

CBG and CBGA given orally at 2 mg/kg twice daily for 2 weeks appears to be very safe for dogs. Physical examination did not reveal any changes in behavior, neurologic status, or activity levels. Serum biochemistry evaluation of the fed state at the 2-week period revealed mild significant elevations in albumin, creatinine, AST, and cholesterol. Creatinine also had mild, yet significant elevation in the fasted state. However, none of these variations were outside of the reference range. More importantly, rises in liver enzymes were absent in our population. ALP in particular, was observed to decrease in both the fasted and fed states. Table 3 shows serum biochemistry results.

When evaluating the complete blood counts, there were mild but significant increases in white blood cell count and neutrophils in both fasted and fed states at the 2-week point compared to the baseline. Platelets and monocytes were significantly increased in the fasted state only. The hematocrit was elevated in the fed state only. While statistically significant alterations occurred, none were clinically relevant as they did not fall outside of the reference ranges. It is unknown if the elevation in white blood cell count and neutrophils is secondary to immune stimulus of CBG or CBGA or if it could be secondary to the oil used as the vehicle for administration. These findings are tenuous since a major limitation of our study was that there was no placebo treatment during this trial therefore the effects on serum chemistry alterations and complete blood counts could be due to the small amount of sesame oil as the base of the hemp supplement provided. Additionally, the dogs used in this study were a small, homogeneous population which may not reflect companion dogs with comorbidities. Complete blood count data is shown in Table 4.

The dogs were followed for bradycardia throughout the initial dosing and over the two-week period of treatment finding no significant changes in heart rate. It may be that the low dose and poor absorption of CBG globally resulted in a lack of α-2 adrenergic stimulation and we can safely assume that the CBGA molecule does not induce bradycardia upon oral absorption in our small cohort. Heart rate data is shown in Table 5.

In conclusion, this study is the first to show that CBG and CBGA hemp extract given orally at 2 mg/kg twice daily administration in sesame oil for 2 weeks is absorbed and retained in the dog. Further studies are necessary to understand elimination kinetics and long-term treatment concerns, however, CBGA is much more absorbable and is retained at a higher serum concentration for a greater length of time, making it more likely to have pharmacologic benefits. Both CBG and CBGA appear to be safe upon short-term evaluation of serum biochemistry, blood count, physical exam and heart rate monitoring in a healthy population of dogs. This specific hemp extract appears to be safe for short-term consumption and caution should be taken when making direct comparisons with other products due to differences in hemp constituents, carrier oils utilized, or other emulsions. Clinical trials to evaluate the pharmacologic benefits of CBG and CBGA in painful, neurologic, and inflammatory conditions are warranted, and a focus on the acidic form may be worthwhile due to the absorption kinetics observed.

TABLE 3
Serum chemistry mean and standard deviation concentrations
(n = 6) at 0 and 2 weeks during the fed and fasted states for
dogs receiving 2 mg/kg body weight of an equal mix of
30 mg/ml CBG and 30 mg/ml CBGA hemp extract.

| Serum | | | | |
|---|---|---|---|---|
| biochemistry | | | | P |
| (reference range) | | Baseline | 2 weeks | value |
| Total Protein | Fed | 5.9 ± 0.2 | 6.1 ± 0.3 | 0.17 |
| (5.5-7.2 g/dL) | Fasted | 5.8 ± 0.2 | 6.1 ± 0.3 | 0.04 |
| Albumin | Fed | 3.7 ± 0.2 | 3.9 ± 0.1 | 0.03 |
| (3.2-4.1 g/dL) | Fasted | 3.6 ± 0.2 | 3.8 ± 0.2 | 0.06 |
| Globulin | Fed | 2.3 ± 0.2 | 2.3 ± 0.2 | 0.79 |
| (1.9-3.7 g/dL) | Fasted | 2.2 ± 0.2 | 2.3 ± 0.2 | 0.14 |
| Creatinine | Fed | 0.7 ± 0.1 | 0.8 ± 0.1 | <0.01 |
| (0.6-1.4 mg/dL) | Fasted | 0.7 ± 0.1 | 0.8 ± 0.1 | <0.01 |
| Urea Nitrogen | Fed | 15 ± 3 | 17 ± 3 | 0.21 |
| (9-26 mg/dL) | Fasted | 14 ± 3 | 15 ± 3 | 0.36 |
| ALT | Fed | 27 ± 4 | 29 ± 6 | 0.11 |
| (17-95 U/L) | Fasted | 26 ± 5 | 30 ± 6 | 0.06 |
| AST | Fed | 27 ± 6 | 32 ± 4 | 0.01 |
| (18-56 U/L) | Fasted | 27 ± 3 | 32 ± 6 | 0.08 |
| ALP | Fed | 43 ± 17 | 38 ± 13 | 0.05 |
| (7-115 U/L) | Fasted | 47 ± 16 | 41 ± 15 | 0.02 |
| Cholesterol | Fed | 209 ± 42 | 239 ± 38 | 0.03 |
| (136-392 mg/dL) | Fasted | 199 ± 47 | 191 ± 36 | 0.32 |
| Glucose | Fed | 91 ± 8 | 106 ± 14 | 0.06 |
| (68-104 mg/dL) | Fasted | 95 ± 7 | 89 ± 8 | 0.1 |
| Total Bilirubin | Fed | 0.1 ± 0.1 | 0.1 ± 0.0 | 0.17 |
| (0.0-0.2 mg/dL) | Fasted | 0.1 ± 0.0 | 0.1 ± 0.1 | 0.17 |
| GGT | Fed | 2 ± 1 | 1 ± 1 | 0.32 |
| (0-8 U/L) | Fasted | 2 ± 1 | 1 ± 1 | 0.45 |

ALT alanine aminotransferase;
AST aspartate aminotransferase,
ALP alkaline phosphatase;
GGT gamma-glutamyltransferase

TABLE 4
Complete blood count means and standard deviations (n = 6)
at 0 and 2 weeks during the Fed and Fasted states for dogs
receiving 2 mg/kg body weight of an equal mix of 30 mg/ml
CBG and 30 mg/ml CBGA hemp extract.

| Complete | | | | |
|---|---|---|---|---|
| Blood Count | | | | P |
| (Reference Range) | | Baseline | 2 weeks | value |
| WBC | Fed | 6.1 ± 0.7 | 7.1 ± 1.0 | 0.01 |
| (5.7-14.2 thou/uL) | Fasted | 6.0 ± 1.0 | 6.8 ± 1.0 | 0.02 |
| HCT | Fed | 54 ± 2 | 57 ± 3 | 0.01 |
| (41-58%) | Fasted | 59 ± 2 | 58 ± 2 | 0.36 |
| Hemoglobin | Fed | 19.5 ± 0.5 | 19.3 ± 0.7 | 0.69 |
| (14.1-20.1 g/dL) | Fasted | 18.5 ± 0.7 | 19.1 ± 0.3 | 0.26 |
| RBC | Fed | 8.2 ± 0.3 | 8.2 ± 0.2 | 0.63 |
| (5.7-8.5 mill/uL) | Fasted | 7.6 ± 0.4 | 8.1 ± 0.3 | 0.06 |
| Platelets | Fed | 270 ± 27 | 296 ± 68 | 0.19 |
| (186-545 thou/uL) | Fasted | 232 ± 13 | 280 ± 24 | <0.01 |
| Neutrophils | Fed | 3.5 ± 0.8 | 4.5 ± 1.1 | 0.01 |
| (3.0-9.6 thou/uL) | Fasted | 3.6 ± 0.7 | 4.2 ± 1.0 | 0.02 |
| Lymphocytes | Fed | 2.1 ± 0.6 | 2.1 ± 0.6 | 0.42 |
| (1.1-4.5 thou/uL) | Fasted | 2.0 ± 0.1 | 2.0 ± 0.5 | 0.91 |
| Monocytes | Fed | 0.2 ± 0.1 | 0.3 ± 0.1 | 0.61 |
| (0.1-1.0 thou/uL) | Fasted | 0.2 ± 0.1 | 0.3 ± 0.1 | <0.01 |
| Eosinophils | Fed | 0.3 ± 0.1 | 0.2 ± 0.1 | 0.46 |
| (0.1-2.1 thou/uL) | Fasted | 0.2 ± 0.0 | 0.2 ± 0.0 | 0.11 |

TABLE 5
Mean and standard deviation (n = 6) for manual heart rate at 9 points in time during
the fed and fasted states for dogs receiving 2 mg/kg body weight of an equal mix of
30 mg/ml CBG and 30 mg/ml CBGA hemp extract.

| Manual | | | | | | | | 1 | 2 | P |
|---|---|---|---|---|---|---|---|---|---|---|
| HR | 0 hr | 1 hr | 2 hr | 4 hr | 12 hr | 24 hr | 48 hr | week | week | value |
| Fed | 107 ± 9 | 108 ± 8 | 113 ± 4 | 111 ± 6 | 105 ± 8 | 108 ± 5 | 107 ± 7 | 108 ± 9 | 109 ± 7 | 0.77 |
| Fasted | 107 ± 7 | 105 ± 11 | 104 ± 11 | 105 ± 8 | 102 ± 11 | 104 ± 7 | 103 ± 11 | 103 ± 8 | 103 ± 9 | 0.14 |

1. A pharmaceutical composition comprising hemp extract and a carrier, wherein the hemp extract comprises:
cannabigerol; and
cannbigerolic acid;
wherein the ratio of cannabigerol to cannabigerolic acid is about 0.2:1 to about 1:0.2.

2. A pharmaceutical composition comprising hemp extract and a carrier, wherein the hemp extract comprises:
cannabigerolic acid;
cannabigerol;
cannabidiol;
cannabidiolic acid;
Δ9-tetrahydrocannabinol; and
cannabichromene;
wherein the ratio of cannabigerol to cannabigerolic acid is about 0.2:1 to about 1:0.2.

3. The pharmaceutical composition of claim 1 or 2, wherein the hemp extract further comprises:
α-pinene;
β-myrcene;
β-pinene;
δ-limonene;
linalool;
β-caryophyllene;
α-humulene;
nerolidol;
guaiol;
caryophyllene oxide; and
α-bisabolol.

4. The pharmaceutical composition of claim 2 or 3, wherein the concentration of Δ9-tetrahydrocannabinol is insufficient to produce a psychotropic effect.

5. The pharmaceutical composition of any one of claims 2-4, wherein the ratio of Δ9-tetrahydrocannabinol to the other cannabinoids is about 1:25.

6. The pharmaceutical composition of any one of claims 2-5, wherein the concentration of Δ9-tetrahydrocannabinol is less than about 10 mg/mL.

7. The pharmaceutical composition of any one of claims 2-6, wherein the concentration of Δ9-tetrahydrocannabinol is less than about 3 mg/mL.

8. The pharmaceutical composition of any one of claims 2-7, wherein the concentration of Δ9-tetrahydrocannabinol is less than about 1 mg/mL.

9. The pharmaceutical composition of any one of claims 2-8, wherein the concentration of Δ9-tetrahydrocannabinol is less than about 0.1 mg/mL.

10. The pharmaceutical composition of any one of claims 2-9, wherein the concentration of Δ9-tetrahydrocannabinol is undetectable.

11. The pharmaceutical composition of any one of claims 2-10, wherein the concentration of Δ9-tetrahydrocannabinol is about 0 mg/mL.

12. The pharmaceutical composition of any one of claims 2-10, wherein the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

13. The pharmaceutical composition of claim 12, wherein the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

14. The pharmaceutical composition of any of one claims 1-13, wherein the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

15. The pharmaceutical composition of claim 14, wherein the hemp extract further comprises:
camphene;
β-ocimene;
eucalyptol;
isopulegol; and/or
nerolidol.

16. The pharmaceutical composition of claim 15, wherein the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

17. The pharmaceutical composition of any one of claims 1-16, wherein the composition is formulated in a carrier.

18. The pharmaceutical composition of claim 17, wherein the carrier is selected from the group consisting of linseed oil, olive oil, fish oil, salmon oil, coconut oil, catnip oil, sesame oil, MCT oil, and grapeseed oil.

19. The pharmaceutical composition of claim 18, wherein the carrier comprises grapeseed oil.

20. The pharmaceutical composition of claim 18, wherein the carrier comprises catnip oil.

21. The pharmaceutical composition of claim 18, wherein the carrier comprises sesame oil.

22. The pharmaceutical composition of any one of claims 1-21, wherein the carrier comprises lecithin.

23. The pharmaceutical composition of claim 22, wherein the lecithin is sunflower lecithin.

24. The pharmaceutical composition of claim 23, wherein the sunflower lecithin is up to 40%.

25. The pharmaceutical composition of any one of claims 1-24, wherein the composition further comprises NF-971P.

26. The pharmaceutical composition of claim 25, wherein the NF-971P is up to 2% weight/volume ratio.

27. A pharmaceutical composition comprising hemp extract and a carrier, wherein the hemp extract comprises:
cannabigerolic acid;
cannabigerol;
cannabidiol;
cannabidiolic acid;
Δ9-tetrahydrocannabinol; and
cannabichromene.

28. The pharmaceutical composition of claim 27, wherein the ratio of cannabigerol to cannabigerolic acid is selected from the group consisting of about 1:100, about 1:50, about 1:10, and about 1:1.

29. The pharmaceutical composition of claim 27 or 28, wherein the ratio of cannabigerol to cannabigerolic acid is about 1:1.

30. The pharmaceutical composition of any one of claims 27-29, wherein the concentration of Δ9-tetrahydrocannabinol is insufficient to produce a psychotropic effect.

31. The pharmaceutical composition of any one of claims 27-30, wherein the ratio of Δ9-tetrahydrocannabinol to the other cannabinoids is about 1:25.

32. The pharmaceutical composition of any one of claims 27-31, wherein the concentration of Δ9-tetrahydrocannabinol is less than about 10 mg/mL.

33. The pharmaceutical composition of any one of claims 27-32, wherein the concentration of Δ9-tetrahydrocannabinol is less than about 5 mg/mL.

34. The pharmaceutical composition of any one of claims 27-33, wherein the concentration of Δ9-tetrahydrocannabinol is less than about 3 mg/mL.

35. The pharmaceutical composition of any one of claims 27-34, wherein the concentration of Δ9-tetrahydrocannabinol is less than about 2 mg/mL.

36. The pharmaceutical composition of any one of claims 27-34, wherein the concentration of Δ9-tetrahydrocannabinol is less than about 1 mg/mL.

37. The pharmaceutical composition of any one of claims 27-35, wherein the concentration of Δ9-tetrahydrocannabinol is about 0 mg/mL.

38. The pharmaceutical composition of any one of claims 27-35, wherein the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

39. The pharmaceutical composition of claim 38, wherein the hemp extract comprises:
about 10-100 mg/mL of cannabigerolic acid;
about 10-100 mg/mL of cannabigerol;
about 1-5 mg/mL cannabidiol;
about 1-5 mg/mL cannabidiolic acid;
less than 1 mg/mL Δ9-tetrahydrocannabinol; and
about 1-4 mg/mL cannabichromene.

40. The pharmaceutical composition of claim 38 or 39, wherein the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

41. The pharmaceutical composition of any one of claims 38-40, wherein the hemp extract comprises:
about 50 mg/mL of cannabigerolic acid;
about 50 mg/mL of cannabigerol;
about 3 mg/mL cannabidiol;
about 3 mg/mL cannabidiolic acid;
less than 1 mg/mL Δ9-tetrahydrocannabinol; and
about 2.7 mg/mL cannabichromene.

42. The pharmaceutical composition of any one of claims 27-41, wherein the hemp extract comprises:
α-pinene;
β-myrcene;
β-pinene;
δ-limonene;
linalool;
β-caryophyllene;
α-humulene;
nerolidol;
guaiol;
caryophyllene oxide; and
α-bisabolol.

43. The pharmaceutical composition of claim 42, wherein the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

44. The pharmaceutical composition of claim 42 or 43, wherein the hemp extract further comprises:
camphene;
β-ocimene;
eucalyptol;
isopulegol; and/or
nerolidol.

45. The pharmaceutical composition of claim 44, wherein the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

46. A dosage form comprising any of the pharmaceutical compositions of any one of claims 1-45 and one or more pharmaceutically acceptable additives, flavoring agents, surfactants, and adjuvants.

47. The dosage form of claim 46, wherein the flavoring agent is selected from the group consisting of peppermint oil, mango extract, beef, poultry, and seafood.

48. The dosage form of claim 46, formulated as a sublingual spray.

49. The dosage form of claim 46, formulated as a water or alcohol soluble solution, or a cream for transdermal application.

50. The dosage form of claim 46, formulated as a gel for buccal or mucosal administration.

51. The dosage form of claim 46, formulated as a powder.

52. The dosage form of claim 46, formulated as a solution for subcutaneous injection.

53. The dosage form of claim 46, formulated as a tablet.

54. The dosage form of claim 46, formulated as a capsule.

55. The dosage form of claim 46, formulated as a hard chewable.

56. The dosage form of claim 46, formulated as a soft chewable.

57. The dosage form of claim 46, wherein the composition is formulated for administration using a nebulizer.

58. The dosage form of claim 46, wherein the composition is formulated for administration using a pet collar.

59. The dosage of claim 46, wherein the composition is formulated as a chew for oral administration.

60. The dosage form of claim 59, where the chew is produced using cold extrusion.

61. The dosage form of claim 60, wherein the weight of the chew is about 0.5-10 g.

62. The dosage form of claim 60, wherein the weight of the chew is about 4 g, about 6 g, about 9 g, or about 10 g.

63. The dosage form of claim 62, wherein the weight of the chew is about 4 g.

64. The dosage form of any one of claims 55-63, wherein the chew comprises:
about 70 mg of cannabigerol;
about 60 mg of cannabigerolic acid;
about 3.2 mg Δ9-tetrahydrocannabinol; and
about 3.6 mg cannabichromene.

65. The dosage form of claim 64, formulated in a carrier for oral administration.

66. The dosage form of claim 65, wherein the carrier is selected from the group consisting of linseed oil, olive oil, fish oil, salmon oil, coconut oil, catnip oil, sesame oil, MCT oil, and grapeseed oil.

67. The dosage form of claim 66, wherein the carrier comprises grapeseed oil.

68. The dosage form of claim 66, wherein the carrier comprises catnip oil.

69. The dosage form of claim 66, wherein the carrier comprises sesame oil.

70. The dosage form of claim 46, formulated for inhalation.

71. A method for treating or reducing pain in a subject in need thereof, comprising administering to the subject a therapeutically effective amount of the composition of any of claims 1-43, or a dosage form of any of claims 46-70.

72. The method of claim 71, wherein the pain is associated with arthritis, post-operative pain, acute pain, joint pain, or multi-joint pain.

73. A method for treating epilepsy in a subject in need thereof, comprising administering to the subject a therapeutically effective amount of the composition of any of claims 1-45, or a dosage form of any of claims 46-70.

74. The method of claim 73, wherein the subject has previously experienced generalized motor seizures or focal seizure episodes.

75. The method of claim 73, wherein the subject has a decrease in the frequency and/or duration of seizures.

76. A method for improving quality of life in a subject with cancer, comprising administering to the subject a therapeutically effective amount of the composition of any of claims 1-45, or a dosage form of any of claims 46-70.

77. The method of claim 76, wherein the subject is receiving L-CHOP or CHOP chemotherapy.

78. The method of claim 76 or 77, wherein the hemp extract, composition or dosage form is administered about every 12 hours starting at week 4 or 5 of doxorubicin treatment.

79. The method of any one of claims 76-78, wherein the cancer is lymphoma.

80. The method of claim 79, wherein the lymphoma is intermediate to high-grade multicentric lymphoma.

81. The method of claim 80, wherein following treatment the subject experiences an absence of lymphoma-associated abnormalities or a decrease in lymph node diameter.

82. The method of any one of claims 76-80, wherein the subject has a body weight >15 kg.

83. The method of any one of claims 76-82, wherein the subject is entering the end of the first cycle of L-CHOP chemotherapy.

84. A method for treating post-operative pain in a subject in need thereof, comprising administering to the subject a therapeutically effective amount of the composition of any of claims 1-45, or a dosage form of any of claims 46-70.

85. The method of claim 84, wherein the subject has undergone tibial plateau leveling osteotomy surgery.

86. The method of claim 85, wherein the subject has been treated with fentanyl and/or a nerve block.

87. The method of any one of claims 76-86, wherein the pharmaceutical composition or dosage form is administered at a dosage of about 0.1-8.0 mg/kg.

88. The method of any one of claims 76-86, wherein the pharmaceutical composition or dosage form is administered at twice the therapeutically effective dosage for one week, and then subsequently administered at a therapeutically effective dosage.

89. The method of claim 88, wherein the therapeutically effective dosage is about 0.1-0.5 mg/kg.

90. The method of claim 88, wherein the therapeutically effective dosage is about 2 mg/kg.

91. The method of claim 88, wherein the therapeutically effective dosage is about 8 mg/kg.

92. The method of any one of claims 76-85, wherein the pharmaceutical composition or dosage form is administered at a dosage of about 1 mg/kg for one week, and then subsequently administered at a dosage of about 0.1-0.5 mg/kg.

93. The method of any one of claims 76-86, wherein the pharmaceutical composition or dosage form is administered at a dosage of about 4 mg/kg for one week, and then subsequently administered at a dosage of about 2 mg/kg.

94. The method of any of claims 74-91, wherein the method results in a therapeutically effective median maximal serum concentration of cannabigerol.

95. The method of claim 94, wherein the median maximal serum concentration of cannabigerol is about 102 ng/mL.

96. The method of claim 94, wherein the median maximal serum concentration of cannabigerol is about 590 ng/mL.

97. The method of any one of claims 76-96, wherein the subject is veterinary.

98. The method of claim 97, wherein the veterinary subject is canine, feline, bovine, porcine, or equine.

99. The method of any one of claims 76-96, wherein the subject is human.

100. A method of achieving an area under the curve from 0 time to 24 hours of between 42.4 and 3048 ng hr/ml for cannabigerol in a subject comprising administering to the subject an effective amount of hemp extract.

101. The method of claim 100, wherein the subject is human, canine or feline.

102. A method of treating or reducing pain in a subject in need thereof, comprising administering a therapeutically effective amount of a pharmaceutical composition comprising hemp extract and a carrier, wherein the hemp extract comprises:
cannabigerol; and
cannabigerolic acid;
wherein the ratio of cannabigerol to cannabigerolic acid is about 0.2:1 to about 1:0.2.

103. A method of treating epilepsy in a subject in need thereof, comprising administering a therapeutically effective amount of a pharmaceutical composition comprising hemp extract and a carrier, wherein the hemp extract comprises:
cannabigerol; and
cannabigerolic acid;
wherein the ratio of cannabigerol to cannabigerolic acid is about 0.2:1 to about 1:0.2.

104. A method of treating cancer in a subject in need thereof, comprising administering a therapeutically effective amount of a pharmaceutical composition comprising hemp extract and a carrier, wherein the hemp extract comprises:
cannabigerol; and
cannabigerolic acid;
wherein the ratio of cannabigerol to cannabigerolic acid is about 0.2:1 to about 1:0.2.

105. A method of improving quality of life in a subject with cancer, comprising administering a therapeutically effective amount of a pharmaceutical composition comprising hemp extract and a carrier, wherein the hemp extract comprises:
cannabigerol; and
cannabigerolic acid;
wherein the ratio of cannabigerol to cannabigerolic acid is about 0.2:1 to about 1:0.2.

106. The method of any one of claims 102-105, wherein the hemp extract further comprises:
cannabigerolic acid;
Δ9-tetrahydrocannabinol; and
cannabichromene.

107. The method of any one of claims 102-105, wherein the hemp extract further comprises four or more of the following:
α-pinene;
β-myrcene;
β-pinene;
δ-limonene;
linalool;
β-caryophyllene;
α-humulene;
nerolidol;
guaiol;
caryophyllene oxide; and
α-bisabolol.

108. The method of claim 106 or 107, wherein the concentration of Δ9-tetrahydrocannabinol is insufficient to produce a psychotropic effect.

109. The method of any one of claims 106-108, wherein the ratio of Δ9-tetrahydrocannabinol to the other cannabinoids is about 1:25.

110. The method of any one of claims 106-109, wherein the concentration of Δ9-tetrahydrocannabinol is less than about 10 mg/mL.

111. The method of any one of claims 106-110, wherein the concentration of Δ9-tetrahydrocannabinol is less than about 5 mg/mL.

112. The method of any one of claims 106-111, wherein the concentration of Δ9-tetrahydrocannabinol is less than about 3 mg/mL.

113. The method of any one of claims 106-112, wherein the concentration of Δ9-tetrahydrocannabinol is less than about 2 mg/mL.

114. The method of any one of claims 106-113, wherein the concentration of Δ9-tetrahydrocannabinol is less than about 1 mg/mL.

115. The method of any one of claims 106-114, wherein the concentration of Δ9-tetrahydrocannabinol is about 0 mg/mL.

116. The method of any one of claims 102-105, wherein the hemp extract comprises:
about 10-100 mg/mL of cannabigerol;
about 10-100 mg/mL of cannabigerolic acid;
less than 1 mg/mL Δ9-tetrahydrocannabinol; and
about 1-4 mg/mL cannabichromene.

117. The method of claim 116, wherein the hemp extract comprises:
about 50 mg/mL of cannabigerol;
about 50 mg/mL of cannabigerolic acid;
less than 1 mg/mL Δ9-tetrahydrocannabinol; and
about 2.7 mg/mL cannabichromene.

118. The method of any of one claims 102-117, wherein the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol;
about 0.02-0.04% guaiol;
about 0.04-0.08% caryophyllene oxide; and
about 0.01-0.04% α-bisabolol.

119. The method of claim 118, wherein the hemp extract further comprises:
camphene;
β-ocimene;
eucalyptol;
isopulegol; and/or
nerolidol.

120. The method of claim 119, wherein the hemp extract comprises:
about 0.02% camphene;
about 0.02-0.03% β-ocimene;
about 0.02-0.05% eucalyptol;
about 0.02% isopulegol; and/or
about 0.02-0.04% nerolidol.

121. The method of claim any one of claims 102-120, wherein the hemp extract comprises 5, 6, 7, 8, 9, 10, 11, 12, 13, 14, 15 or more of the following: α-pinene, β-myrcene, β-pinene, δ-limonene, linalool, β-caryophyllene, α-humulene, nerolidol, guaiol, caryophyllene oxide, α-bisabolol, camphene, β-ocimene, eucalyptol, isopulegol, and nerolidol.

122. The method of any one of claims 102-121, wherein the composition is formulated in a carrier.

123. The method of claim 122, wherein the carrier is selected from the group consisting of linseed oil, olive oil, fish oil, salmon oil, coconut oil, catnip oil, sesame oil, MCT oil, and grapeseed oil.

124. The method of claim 123, wherein the carrier comprises grapeseed oil.

125. The method of claim 123, wherein the carrier comprises catnip oil.

126. The method of claim 123, wherein the carrier comprises sesame oil.

127. The method of any one of claims 102-126, wherein the composition comprises nepetalactone.

128. The method of any one of claims 102-127, wherein the composition comprises taurine.

129. The method of any one of claims 102-128, wherein the composition is formulated for administration using a nebulizer.

130. The method of any one of claims 102-128, wherein the composition is formulated for administration using a diffuser.

131. The method of any one of claims 102-128, wherein the composition is formulated for administration using a pet collar.

132. The method of any one of claims 102-128, wherein the composition is formulated as a pet food for oral administration.

133. The method of any one of claims 102-128, wherein the composition is formulated as a chew for oral administration.

134. The method of claim 133, wherein the weight of the chew is about 0.5-10 g.

135. The method of claim 134, wherein the weight of the chew is about 4 g, about 6 g, about 9 g, or about 10 g.

136. The method of claim 135, wherein the weight of the chew is about 4 g.

137. The method of any one of claims 133-136, wherein the chew comprises:
about 70 mg of cannabigerol;
about 60 mg of cannabigerolic acid;
about 3.2 mg Δ9-tetrahydrocannabinol; and
about 3.6 mg cannabichromene.
